(19) United States
(12) Patent Application Publication
(10) Pub. No.: US 2026/0185981 A1
(43) Pub. Date: Jul. 2, 2026

(54) MEANS AND METHODS FOR DETERMINING CELLULAR AVIDITY

(71) Applicant: LUMICKS CA HOLDING B.V., Amsterdam (NL)

(72) Inventors: Marinus Theodorus Johannes VAN LOENHOUT, Amsterdam (NL); Trillian Ashley GREGG, Amsterdam (NL); Serena Alba DAVOLI, Amsterdam (NL); Mattijs DE GROOT, Amsterdam (NL)

(21) Appl. No.: 18/863,877

(22) PCT Filed: May 12, 2023
(86) PCT No.: PCT/EP2023/062720

§ 371 Date: Nov. 7, 2024

(30) Foreign Application Priority Data
May 13, 2022 (EP) 22173282.9
May 13, 2022 (EP) 22173311.6

(51) Int. Cl. G01N 33/50

(52) U.S. Cl. CPC G01N 33/5032; G01N 33/505

(57) ABSTRACT

The current invention relates to improved methods utilizing cellular avidity, said methods involving applying a force such that cell-cell bonds can be broken, and furthermore detecting the presence or absence of a marker associated with synapse formation in cells that remain bound thereafter. These methods are highly useful in determining cellular avidity and also for use in sorting cells of interest and the like. These methods are also highly useful in screening assays e.g. for screening effector cells or cell therapy candidates.

### INTRODUCTION

Cellular avidity, i.e. a measure representative of the binding strength between cells, can be determined by allowing cells to interact to form a bond, and assessing the strength required to have the bound cells detach from each other. For example, with a device from LUMICKS (e.g. the z-Movi® device), the force at which a cell detachment event occurs can be determined, and the combined measurement of cell detachment events when a force ramp is applied provides for a cellular avidity plot representing cellular avidity, e.g. the more cells remain attached at the end of the force ramp, the higher cellular avidity is.

Cellular avidity between cells of interest like T cells and target cells like cancer cells, e.g. T cells carrying a TCR specific to a defined target antigen presented with MHC on a cancer cell, involves in addition to MHC and TCR interaction further co-receptor/ligand interactions between the cells and/or further cellular components that are involved in synapse formation. Cells in general often may have the tendency to interact such that cells are attached to each other. Such type of attachment is different from potentially highly specific interactions which are largely driven by TCR/MHC interactions and the like. Such other forms of general and less specific interactions tend to result in much lower cellular avidities as compared with a TCR/MHC interaction or the like.

In order to differentiate between specific and aspecific interactions that can occur between different cells (e.g. an effector cell and a target cell), in the art, control cells are provided, e.g. a cell with a control TCR not specifically interacting with the target cells or a control target cell not displaying the antigen, and cellular avidity determined therewith. This way, an assessment can be made for cellular avidity related to a specific interaction of interest such as an MHC-TCR interaction. For example, one can determine a ratio of, or difference between, a determined cellular avidity of a T cell presenting TCR with a target cell, and of a T cell presenting a control TCR with a target cell. This way, a measure of cellular avidity related to specific interactions can be provided allowing to compare e.g. different TCRs.

The current inventors now sought to provide for improved means and methods for assessing cellular avidity and its use.

### SUMMARY OF THE INVENTION

The current inventors working with cellular avidity and its use, sought to improve cellular avidity measurements further. The inventors realized that although using control cells is highly helpful, a control cell with a different TCR may still have different aspecific interactions as compared with the effector cell of interest, and a control target cell, in particular in the field of cancer, may present other antigens as well. Furthermore, suitable control cells may not always be available and comparing differences in cellular avidity between cells of interest and control cells may not be sufficient in all cases to identify the most interesting cells. Hence, the current inventors sought for improved means and methods to assess cellular avidity in a way to distinguish further between specific and aspecific interactions.

Highly advantageously, by including in cellular avidity measurements i.e. measuring detachment events while exerting a force, the determination of markers, e.g. cell surface molecules or intracellular factors involved in cell-cell attachment, in particular involved in synapse formation, an improved cellular avidity score can be provided that more accurately represents cellular avidity attributable to specific interactions. This way, the effect of exerting a force on cell-cell interactions can be further differentiated. This way, cells that remain bound after applying a force in a cellular avidity measurement, can be differentiated with regard to whether or not these cells have or have had a specific TCR/MHC interaction or the like, or rather represent a more general aspecific cell-cell interaction.

Highly advantageously, the cells that have remained bound after applying the force can simply be resuspended, providing for a mixture of cells comprising singlets, i.e. single cells, and comprising doublets, i.e. a target cell and effector cell bound to each other. The terms singlet and doublet are terms commonly used in fluorescent activated cell sorting analysis or the like, and singlet cells and doublet cells can easily be separated from each other utilizing FACS. This mixture can easily be obtained and subsequently be processed for analysis in accordance with the invention to thereby determine markers associated with synapse formation in order to provide for a cellular avidity score. By first applying a force, to remove non-binding cells and aspecific binding cells, much less cells are provided for subsequent analysis. Such may be highly advantageous, e.g. when utilizing methods such as one or more of (single-cell) sequencing, FACS analysis, staining, and sorting.

Furthermore, the inventors also found that the cells that have remained bound, i.e. effector cells with target cells, after applying the force can be enzymatically treated, e.g. using trypsin, thereby obtaining a mixture substantially comprised of singlets, i.e. single cells. Hence, this allows to easily obtain all the cells after the force has been exerted allowing for subsequent analysis utilizing methods known in the art.

In any case, cells that have been involved in cell-cell interactions and remain after applying a force can be obtained and subsequently analysed with regard to markers associated with synapse formation. By analysing such markers, in at least cells that have formed a bond with each other, a cellular avidity score can be provided taking into account functional interactions, e.g. specific interactions that resulted in a synapse. This way, advantageously, it may no longer be required to determine cellular avidity of control cells. Moreover, any uncertainty on whether or not control cells actually represent a proper control and provide for a relevant control avidity score, may be avoided. Furthermore, by using such a marker in conjunction with assessing cellular avidity (rare) cells of interest may be more readily identified in a heterogeneous cell population and/or cell potency may be more accurately determined. The identification of cells that remain bound to each other after a force was exerted thereon, and for which a marker associated with synapse formation is detected, is what is required to highly improve determining cellular avidity in accordance with the invention.

This way, for effector cells carrying receptors, functional cellular avidity scores can be determined. Moreover, more in depth information on the stage of synapse formation can be included in the cellular avidity score as well. For example, by studying markers associated with the early phase of synapse formation and/or late phase of synapse formation. This way, highly valuable cellular avidity scores can be provided which represent much improved and better-defined cellular avidity scores.

Furthermore, identifying such markers, in at least cells that have formed a bond with each other, is highly useful in screening and discovery methods. For example, in identifying effector cells having a receptor of interest. Alternatively, the means and methods in accordance with the invention also allow for identifying novel markers that are associated with synapse formation. When utilizing markers associated with synapse formation in the methods in accordance with the invention, it may no longer be required to determine cellular avidity of control cells and compare therewith. Moreover, any uncertainty on whether or not control cells actually represent a proper control and provide for e.g. a relevant control cellular avidity score, may be avoided. The identification of cells that remain bound to each other after a force was exerted thereon, and for which a marker associated with synapse formation is detected, is what is required to highly improve screening and discovery methods utilizing cellular avidity. Moreover, for effector cells, cellular avidity scores can be determined including more in depth information (on the stage) of synapse formation as well. This way, highly valuable means and methods utilizing cellular avidity are provided.

### FIGURES

FIG. 1. Schematic outlining the forces which play a role in cellular avidity measurements involving cells attached to a surface. Cells are provided attached to a surface. In the scenario depicted, a force Fₘ is exerted away from the attached cells, i.e. perpendicular from the surface. Of course, the force may be parallel relative to the surface as well, e.g. in case of a shear force. The force, Fₘ, that can be exerted is restricted by the force at which the cells attached to the surface will detach therefrom (i.e. F_{c}), which means that Fₘ<F_{c}. With regard to a cellular avidity measurement, the forces that are required for cells (depicted as white cells) that have interacted with the attached cells (depicted as grey cells) to move away from the attached cells determine the outcome of a cellular avidity measurement. Ideally, the force required to have aspecifically bound cells move away, i.e. Fₐ, is lower than F_{c}, and the force required to have specifically bound cells that formed e.g. an immune synapse, i.e. F_{b}, would be higher than Fₘ and Fₐ. In case aspecifically bound cells require a force Fₐ which is larger than Fₘ or F_{c}, such aspecifically bound cells will remain and may confound cellular avidity measurements. As often occurs in cellular avidity measurements, as exemplified e.g. by background binding observed with control measurements, Fₐ can be larger than F_{c} and/or Fₘ, resulting in retaining at least part of the cells bound aspecifically at the end of an applied force, e.g. a force ramp. This means that bound cells at the end of a cellular avidity measurement, e.g. with an applied force ramp, often have either a synapse or are bound aspecifically to the cells attached to the surface. This means that at the end of a cellular avidity measurement effector cells that remain bound have formed a synapse or represent effector cells that have a (relatively) strong aspecific interaction. These force principles underly i.a. the means and methods involving determining cellular avidity. It is understood that the effector cells instead of the target cells may be attached to the surface. It may be preferred to have target cells attached to the surface, e.g. because these types of cells may more easily sufficiently attach to a surface such that a cellular avidity measurement can be performed.

FIG. 2. Scheme outlining a process for assessing cellular avidity. In a) effector cells (white, N) are provided, and target cells (grey, M) are provided attached to a surface. Subsequently, in b) the effector cells are provided to interact with the target cells attached to the surface. After incubation, some effector cells bind to target cells. Next, a force (Fₘ) is exerted away from the surface, away from the target cells, such that effector cells that have not bound to the target cells move away from the surface. In addition, the force may cause bound effector cells to detach from the target cells. The cells that detach or move away, c), can be collected for subsequent analysis, e.g. for determining the number of cells N(i) but may also be disposed of as these may be of less interest. After the force has been exerted, the surface, shown in d), comprises target cells, and target cells with effector cells, bound thereto. Some of the effector cells have a specific interaction and form a synapse (shown with dashes) and others have an aspecific binding (no dashes). The cells can subsequently be obtained therefrom, e.g. via mechanically removing the cells from the surface and suspending these cells, thereby obtaining, in e) a mixture comprising substantially singlets (primarily of target cells, M(i)), and doublets (effector cells N(ii) and (iii), attached to target cells, M(ii) and (iii) respectively), which can be further separated, e.g. by sorting 20 utilizing FACS or the like. This way, of different fractions populations can be obtained as shown in f) such as: effector cells N(i), target cells M(i), and effector cells attached to target cells (effector cells N(ii) and N(iii), attached to target cells, M(ii) and M(iii) respectively), wherein the effector cells attached to target cells that formed a synapse (shown with dashes) can be identified by determining the presence of a marker. Of course, the marker can also be determined having the cells remained attached to the surface as shown in d), e.g. using microscopy methods. Finally, as depicted in g) with formulas and calculations exemplified (which are non-exhaustive) the numbers determined of a fraction can be used to calculate a cellular avidity score (CA). A cellular avidity score which does not take into account synapse formation is exemplified by CA, whereas CA′-CA″″ represent cellular avidity scores taking into account synapse formation. As described above, the target cells (grey) were attached to a surface and the effector cells interacted therewith (white). Although this constellation may be preferred, the reverse constellation may also be an option, i.e. wherein the effector cells (grey) were attached to a surface and the target cells provided to interact therewith (white). As such a scenario may result in a substantial amount of effector cells being present, this may be less preferred, e.g. when these are to be analysed. Subsequent formulas and calculations as depicted in g) with which cellular avidity scores, if of interest, can be determined may equally apply.

FIG. 3. Obtained singlets and doublets may subsequently e.g. be chemically or enzymatically treated, e.g. with trypsin, to obtain single cells. These single cells may subsequently be analysed, e.g. individually, e.g. via single cell sequencing, staining with a label, or the like. Cells may be fixed before the subsequent analysis. Also, obtaining single cells may allow to separate effector cells from target cells for a subsequent analysis. Such a chemical or enzymatic treatment may be performed on cells obtained from the surface, after the force has been exerted, as depicted in A) which may be on a fraction of doublets separated from singlets. Alternatively, or in addition, e.g. in scenarios wherein mechanically collecting the cells may be challenging, one may also contemplate to perform such a chemical or enzymatic treatment in situ, i.e. on the cells attached to the surface, and simply collect a mixture of cells, as depicted in B). In any case, highly enriched fractions can be obtained, wherein in the target cell and/or effector cell markers can be identified associated with synapse formation (indicated with dashes), as depicted in C). In case the number of cells having such a marker is determined it may allow to determine a cellular avidity score more accurately.

FIG. 4. Shows a calcium flux time plot of an effector cell-target cell synapse bond. The arrow indicates calcium oscillations from minute 7 onwards.

FIG. 5. Schematic showing resuspension of cells obtained after applying a force. After the force has been applied, (a), the cells can be collected, e.g. via resuspension, by forceful flushing causing high shear force, or other mechanical means (b). The obtained cells may be forced e.g. through a nozzle (c), which induces a differential force on cells bound to each other, i.e. the force exerted on the white cell is higher (Fq) than the force on the grey cell (Fr), the net force exerted being Fq−Fr=Fs. If the net force is large enough to break a cell-cell bond, the cell-cell bond will rupture. This can result in substantially aspecific cell-cell bonds to break, while cell-cell synapse bonds remain (d). In such a process, the same principles as depicted in FIG. 1 apply, the difference being that the differential force that is exerted is not restricted by the strength of the attachment of the cells to the surface, nor is it required to have the cells attached. This means that the net force Fs applied can be larger than the force that would be required to detach cells attached to the surface Fc (See FIG. 1). Furthermore, it can also be envisioned to not have the cells attached in a) and solely apply the differential force instead.

FIG. 6. FACS analysis of resuspended fractions treated with trypsin or with a PBS control. A) plots event counts versus FITC-A intensity for PBS control, and in B), for the population of the gated FITC-A counts depicted in A), FITC-A intensity was plotted against the intensity of the PB450 channel. Likewise, C) plots event counts versus FITC-A intensity for the trypsin treated fraction, and in D), for the population of the gated FITC-A counts depicted in C), FITC-A intensity was plotted against the intensity of the PB450 channel.

### DETAILED DESCRIPTION

As said, the current inventors in working with means and methods for determining cellular avidity now realized that when in such means and methods, the presence or absence of a marker associated with synapse formation is determined, this can highly advantageously improve such means and methods.

In a first embodiment, a method is provided for determining the cellular avidity of an effector cell carrying a receptor, capable of binding target cells, comprising the steps of:
a) providing effector cells carrying a receptor;
b) providing target cells;
wherein the effector cells or the target cells are attached to a surface;
c) contacting the effector cells with the target cells to allow the cells to interact with each other;
d) applying a force away from the cells attached to the surface, such that at least part of the cells bound to the cells attached to the surface move away therefrom;
e) detecting effector cells that have remained bound with the target cells and attached to the surface after applying the force and having a marker associated with synapse formation, and determine the number of effector cells and/or target cells associated with said marker; and,
f) assigning a cellular avidity score based on the determined number of effector cells and/or target cells associated with said marker.

Hence, in the first step of the method, effector cells carrying a receptor are provided. In accordance with the invention, effector cells carrying a receptor include effector cells of the immune system that can exert an effect, via the receptor. For example, a T cell carrying a T cell receptor can bind an antigen on a cancer cell, upon which it can e.g. exert a cytotoxic effect and kill the target cell. Effector cells can be derived from nature, e.g. obtained from a host, and can also include genetically modified cells wherein e.g. a receptor in particular useful is provided to an effector cell.

Next, target cells are provided. Target cells in accordance with the invention are the cells on which the effector cells are to exert an effect, e.g. bind therewith and trigger an immune reaction thereto. Target cells include cancer cells presenting an antigen. An antigen may be presented by MHC, i.e. HLA in humans, which are specialized receptors that present peptides e.g. derived from digested proteins expressed by the cell (e.g. usually 8-11 amino acids in length for MHCI). An antigen may also be a protein or other biomolecule that is presented on the surface of a cell, e.g. epidermal growth factor receptors or checkpoint proteins, which in the case of cancer cells are overexpressed therewith providing a differentiating feature. Target cells may also include cells expressing auto-antigens, e.g. known to be involved in autoimmunity diseases or cells infected with a pathogen, e.g. a virus.

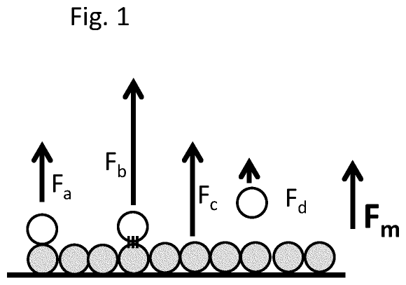

Subsequently, either the target cells or the effector cells are attached to a surface. It is understood that surfaces for attaching cells may be any surface suitable for attaching cells. Suitable surfaces for attaching cells include plastic or glass surfaces. These surfaces may be coated e.g. with a protein to attach cells to the surface, such as poly-L-lysine or the like. The attachment of the cells to the surface is such that the strength of the binding to the surface is sufficient to have the cells remaining attached when applying a suitable force (see i.a. FIG. 1).

The effector cell carrying the receptor is capable of binding target cells, or effector cell may be analysed for its capability of binding target cells, e.g. in screening and discovery methods as envisioned herein. It is understood this capability of binding target cells includes a specific interaction that can induce synapse formation, i.e. the effector cell is to bind to a target cell and exert an effect thereon, e.g. induce target cell killing.

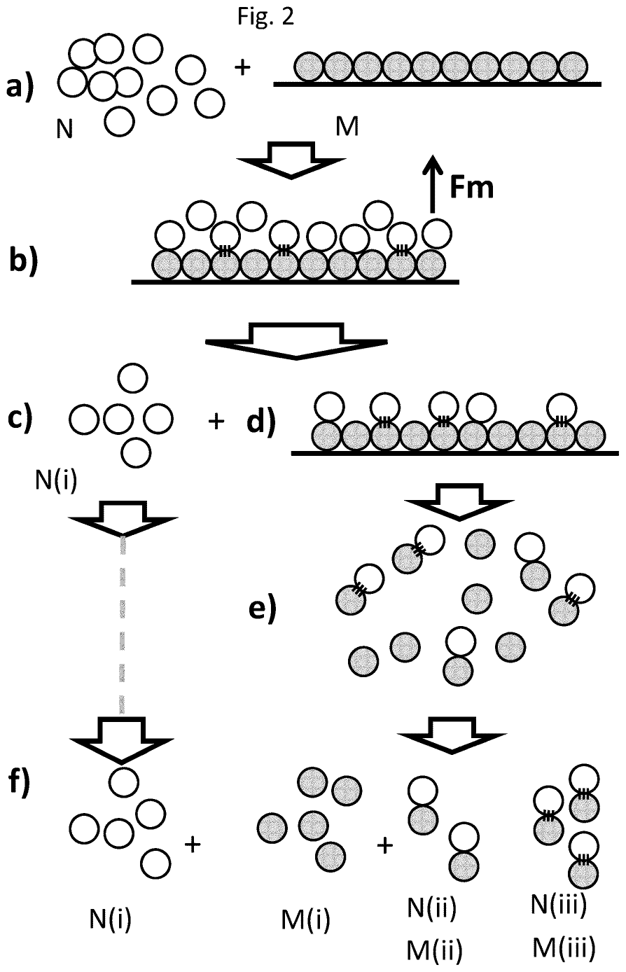

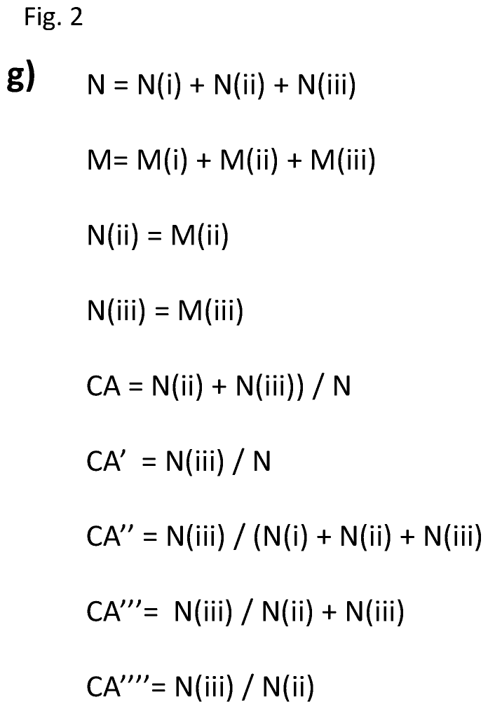

In the next step, effector cells and the target cells are contacted with each other to allow the cells to interact. This step is such that the effector cells will have sufficient time to interact with target cells and can form a bond, including synapses (see i.a. FIG. 2). It is understood that a cell-cell interaction may not always result in a cell-cell bond, which can be a synapse or aspecific bond, the contacting step is such that cell-cell bonds can be formed and appropriate conditions therefore are selected. It is understood that because the conditions are selected such that a synapse can be formed, this necessarily implies that aspecific cell-cell bonds are allowed to be formed at the same time.

Once the effector cells have had contact with the target cells, and have had the opportunity, if possible, to form a synapse, in a subsequent step a force is applied away from the cells attached to the surface, such that at least part of the cells bound to the cells attached to the surface, and unbound cells as well, move away from the cells attached to the surface. This way, target cells are obtained with effector cells bound thereto, which are attached to the surface, wherein the exerted force was not sufficient to break cell-cell bonds. Of course, cells that are attached to the surface, to which no subsequent cells are bound, remain as well.

As said, the force is applied in a direction away from the attached cells. It is understood that in this step, the force applied may be perpendicular (in the direction of z-axis) to the surface (x,y) to which cells are attached, for example when a centrifugal force or acoustic force is applied. The force may also be lateral (in the direction of the x-axis or y-axis relative to the surface), for example when a shear force is applied. In any case, the force is applied and is controlled such that a defined force is exerted on the cells that interacted with the attached cells. It is understood that the force that is exerted on the cells interacting with attached cells is to be substantially equal, such can be achieved e.g. when using a flat surface. Other suitable surface shapes may be used (e.g. a tube with exerted concentrical force or laminar flow force in the direction of the length of the tube), as long as the force exerted can be substantially equal at a defined surface area to which cells are attached, such a surface shape may be contemplated.

The cells that are attached are preferably attached to a glass or plastic surface, preferably a surface in a chip, which allows for detection of cells e.g. via microscopy or other means. The applied force required to move a cell away from an attached cell preferably can be detected, e.g. via microscopy or other means, to which may be referred to as a cell detachment event or cells moving away. This way, cells moving away can be monitored and counted. It may be advantageous and convenient to use microscopy, with which bound cells can be identified and quantified and cells moving away can be likewise monitored and quantified, also allowing for detection of markers. For example, the z-Movi® device as available from LUMICKS which applies an acoustic force may be well equipped to do so. Likewise, similar devices may be provided with microscopy or other means to quantify cells, detect markers and bound cells and/or cells moving away, and also utilizing e.g. shear force or centrifugal forces instead of acoustic force.

The effector cells that have remained bound with the target cells and attached to the surface have subsequently detected therein or associated therewith the presence or absence of a marker associated with synapse formation. It is understood that with regard to the effector cells that have remained bound with target cells and attached to the surface, and that have a marker associated with synapse formation determined, means that either the determined marker is from the effector cell or from the target cell. It is also understood that one or more markers may be determined. It may also be contemplated that one or more markers are determined of the effector cell and one or more markers are determined of the target cells. As long as the one or more markers associated with synapse formation that are determined can be assigned to an effector cell bound to a target cell, such a marker determination can be contemplated. Suitable means and methods to determine markers in an effector cell or target cell are described herein further below.

It is understood that a synapse is a specialized structure that forms when the plasma membranes of two cells come into close proximity to transmit signals. Cells of the immune system form synapses that are essential for cell activation and function. Lymphocytes such as T cells, B cells and natural killer (NK) cells form synapses that can be referred to as immunological synapses. Such a synapse typically forms between immune effector cells and target cells, e.g. cells presenting an antigen. A non-limiting example is e.g. a T cell or a CAR-T cell and a cancer cell. The formation of synapses between e.g. an effector cell and a target cell, for example an APC (antigen presenting cell), is a hall-mark event and signals the presence of specific interactions (i.e. the specific interaction between, for example, a TCR or CAR and an antigen recognized thereby) between the effector cell and the target cell that are involved in the formation of such immunological synapses.

In the case of a T cell, a synapse can be formed between the lymphocyte and antigen-presenting cells (APCs) during the recognition of the peptide antigen-major histocompatibility complex (pMHC) ligand by the T cell antigen receptor (TCR). The TCR and pMHC are both membrane-bound, so the TCR will only be triggered by its ligand at the interface between T cells and APCs. A synapse can be observed at the T cell-APC interface as concentric rings by confocal microscopy, often referred to as “bull's eye” (Huppa, J. B., & Davis, M. M. (2003). T cell-antigen recognition and the immunological synapse. Nature Reviews Immunology, 3(12), 973-983). These rings were named the central, peripheral, and distal supramolecular activation cluster (i.e. respectively cSMAC, pSMAC and dSMAC). The TCR has been reported to be present in the cSMAC, whereas other lymphocyte specific proteins such as lymphocyte function-associated antigen-1 (LFA-1), are integrated into the pSMAC ring that surrounds the TCR. The formation of this ringed structure (“bull's eye”) is however not universal and other formations such as “multifocal immunological synapses” between T cells and dendritic cells, or the like, have been described.

The immunological synapse can be considered to be any structure formed at the interface resulting from a functional and specific effector-target cell interaction, such as for example T cell-APC contacts. Markers associated with effector cells and synapse formation include one or more of CD43, CD44, CD45, LFA-1, Talin, F-actin, ZAP70, CD2, CD4, CD8, CD3, CD28, PD-1, ICOS, and TCR. Markers of target cells include one or more of ICAM-1 (associates with LFA-1), CD48/58 (interacting with CD2) CD80/CD86 (interacting with CTLA-4 and CD28), PDL1/PDL2 (associating with PD-1), and MHC presenting the antigen (that specifically interacts with the TCR). These markers, and concentrations thereof, may be detected e.g. with fluorescent labels at the interface between effector cell and target cell, or intracellularly in close proximity to the synaptic interface.

For example, markers of effector cells that have been associated with dSMAC are CD43, CD44, CD45. The effector cell markers LFA-1, Talin, F-actin, CD2, CD4 and CD8 have been associated with pSMAC. The markers CD3, CD28, PD-1, ICOS, and TCR of effector cells have been associated with cSMAC. Markers that have been associated with a synapse on a target cell are ICAM-1 (associates with LFA-1), CD48/58 (interacting with CD2) CD80/CD86 (interacting with CTLA-4 and CD28), and PDL1/PDL2 (associating with PD-1), and of course an MHC presenting the antigen (that specifically interacts with the TCR). These markers have been shown to be associated with synapses in a TCR-MHC interaction. Likewise, cell engagers (such as a bispecific antibody that e.g. binds CD3 on an effector cell with one arm and with the other arm an antigen on a target cell) which engage an effector cell with a target cell, can trigger the formation of a similar synapse structure as observed with a classic TCR-MHC interaction.

With regard to CARs of e.g. CAR T cells, these are chimeric antigen receptors that are to mimic a TCR or the like. CARs are engineered. The first generation of CAR were provided with an antigen recognition part often an antibody derived region (e.g. a scFv) fused to a transmembrane region and intracellular region of e.g. a CD3 ζ-chain. Later generations combined intracellular signalling domains from various costimulatory protein receptors (e.g., CD28, 41BB, ICOS) incorporated in the cytoplasmic tail of the CAR to enhance further signalling. Further generations also incorporated in their design an inducible release of transgenic immune modifiers, such as IL-12, to shape the tumor environment by augmenting e.g. T cell activation, attracting and activating innate immunity. As CARs often have antibody variable regions incorporated, these can target e.g. receptors themselves that are presented at the surface of a cell (e.g. Her2, PD-1 etc.), or can also target antigens presented by MHC, derived e.g. from proteins intracellular processed by the ubiquitin-proteasome system. Such peptides presented by MHC include proteins that are processed internally and presented by MHC, which can be derived from receptors, secreted proteins, intracellular proteins or internalized proteins. Synapses formed between CARs and target cells may not provide a classical bull's-eye like structure with a well-characterized SMAC domain, but may result in less organized patterns. Multiple CAR microclusters form and signalling molecules associate, which are dispersed in the center of the synapse interface.

Synapse formation is a spatiotemporal process that starts e.g. by TCR binding to MHC or binding of an antigen with CAR or cell engagement, and subsequent phosphorylation of the cytosolic tails of CD3 resulting in a triggered state. This sets of a cascade of processes that result in an activated T cell state, which also depends on the effector cell type and its phenotypic state. Key processes include: calcium signalling, internal cell structure and/or cytoskeleton changes of effector cells, involving F-Actin, Talin, and changes in microtubules, centrosomes, lytic granules, nucleus position, and mitochondrial location. Transactivation of adhesion molecules, cytokine and marker expression. IFNγ, granzyme and perforin may be released by effector cells to thereby induce i.a. target cell killing. Also, in addition, in target cells apoptotic markers can be found, including ICAM-1 clustering, phosphatidyl translocation, mitochondrial depolarization, caspase-3 activation and DNA fragmentation. Hence, various stages of specific effector cell and target cell interaction and immune activation can be detected.

In any case, synapse formation, or a synapse can be determined by staining, i.e. with fluorescent labels, ligands, antibodies, or probes, or the like, which may be used on live cells or on fixed cells targeting the mechanisms involved in T cell activation and/or synapse formation. Sequencing based methods may also be used to identify activated T cells and thereby associate mRNA levels with the formation of stable synapses. T cell activation leads to changes in mRNA stability and expression. E.g. increases in expression of cytokine or secretory transcripts (IL2, IFNγ, granzyme, perforin) and proliferation pathways and either bulk or single-cell RNA sequencing may be used to detect these changes and correlate these to the number of synapses formed. Hence, synapse formation and effector cell activation, including its stages, can be detected by means of staining, fluorescent labelling, sequencing and the like.

For example, as indicated below and as described in the examples herein, an effector cell-target cell pair that remains bound after the force has been exerted, may be isolated and subsequently stained or sequenced to detect synapse formation. The cells may be optionally fixated, e.g. with methanol, glutaraldehyde or para-formaldehyde or the like. It is also understood that it may not be necessary to isolate the cells, but one may e.g. stain effector cell-target cell pairs in situ.

As said, either the effector cells or the target cells may be attached to a surface. It may be preferred, in one embodiment, to have the target cells attached to a surface. This is because e.g. target cells may be more easily attached to a suitable surface.

Hence, of the effector cells that have remained bound with target cells and attached to the surface, a marker associated with synapse formation, can thus be determined, and the number of effector cells and/or target cells associated with said marker is determined as well. As a synapse is formed between effector cells and target cells, the number of effector cells or target cells associated with said marker each represents the number of synapses formed, hence, determining either number may suffice. Off course, one may also determine the number for both effector cells and target cells, e.g. from a quality control perspective, as their numbers should be substantially similar.

Subsequently, based on the determined number of effector cells and/or target cells associated with said marker, a cellular avidity score can be determined. The higher the number of cells associated with said marker, the higher the cellular avidity score. For example, which is the case if the cellular avidity score is determined in relation to the number of cells contacted and/or the number of cells not associated with the marker.

With regard to the cellular avidity score as determined herein in accordance with the invention, it is understood that this takes into account the number of cells that have remained attached after applying the force and which also takes into account one or more markers associated with synapse formation. Hence, as already outlined above, as compared with a cellular avidity score which only takes into account detachment of cells and applying a force, the cellular avidity score in accordance with the invention may provide for a more accurate cellular avidity score which is more reflective of the function of effector cells, i.e. forming synapses with target cells. Hence, the cellular avidity score as determined herein in accordance with the invention may also be referred to as a functional cellular avidity score or a synaptic cellular avidity score.

Alternatively, as the result of forming a synapse between an effector cell and a target cell is cell killing, the cellular avidity assay in accordance with the invention may also be an alternative to a potency assay. By measuring markers associated with synapse formation, the most potent, i.e. most strong, cell-cell interaction is determined. Hence, the cellular avidity as determined herein may also be referred to as a cellular avidity potency, and the cellular avidity score referred to as cellular avidity potency score.

As said, either the effector cells or the target cells may be attached to a surface. It may be preferred, in one embodiment, to have the target cells attached to a surface. This is because e.g. target cells may be more easily attached to a suitable surface. Hence, in one embodiment, a method is provided for determining the cellular avidity of an effector cell carrying a receptor, capable of binding target cells, comprising the steps of:
a) providing effector cells carrying a receptor;
b) providing target cells attached to a surface;
c) contacting the effector cells with the target cells to allow the cells to interact with each other;
d) applying a force on the effector cells away from the target cells, such that at least part of the cells carrying the receptor detach and/or move away from the target cells;
e) detecting effector cells bound to the target cells after applying the force and having a marker associated with synapse formation, and determine the number of effector cells and/or target cells associated with said marker; and
f) assigning a functional cellular avidity score based on the determined number of effector cells and/or target cells associated with said marker.

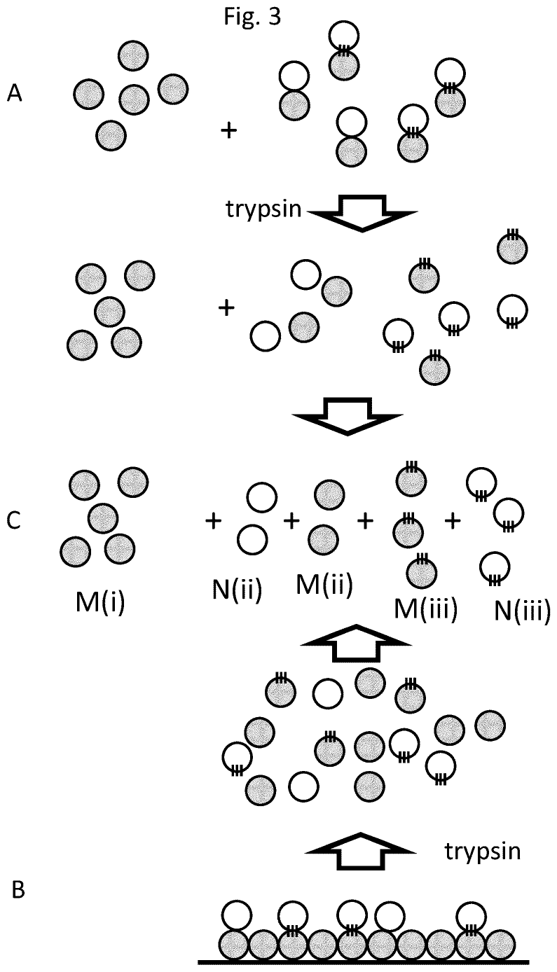

In a further embodiment, the number of effector cells bound to target cells is determined after the step of applying a force. This number reflects the number of effector cells that formed a bond with target cells which is useful e.g. in determining a cellular avidity score in accordance with the invention. For example, one can calculate the ratio of the number of effector or target cells that formed a synapse to the number of effector cells or target cells that remained bound to each other after exerting a force as a cellular avidity score. Cellular avidity can be expressed in different ways taking into account the number of cells that have formed a synapse, relative to the number of cells that interacted with the attached cells, or relative to the number of cells that remained bound after exerting a force, or the number of cells that did not remain bound after exerting a force and the number of cells that bound but did not form a synapse. As depicted in FIGS. 2 and 3, different fractions at different steps of the methods can be collected and the number of cells as determined from such fractions or known (e.g. of a starting fraction) can be used to determine a cellular avidity score in accordance with the invention, taking into account the number of cells associated with synapse formation (e.g. fractions (iii) in FIG. 2). In one embodiment, a cellular avidity score is determined as outlined in FIG. 2.

Hence, in one embodiment, a method is provided, wherein the cellular avidity score is determined by calculating the ratio of the number of effector cells carrying the receptor with the marker associated with synapse formation to the number of effector cells bound to the target cells after step d); or, wherein the functional cellular avidity score is determined by calculating the ratio of the number of target cells associated with said marker associated with synapse formation to the number of effector cells bound to the target cells after step d).

In another embodiment, the functional cellular avidity score is determined by calculating the ratio of the number of effector cells carrying the receptor with the marker associated with synapse formation to the number of effector cells that have interacted with the target cells in the contacting step c); or, wherein the functional cellular avidity score is determined by calculating the ratio of the number of target cells associated with said marker associated with synapse formation to the number of target cells that have interacted with the effector cells in the contacting step c).

In yet another embodiment, a method is provided herein wherein the functional cellular avidity score is determined by calculating the ratio of the number of effector cells carrying the receptor with the marker associated with synapse formation to the number of effector cells that have interacted with the target cells in the contacting step c) and that did not remain bound with the target cells after applying the force in step d) and the number of effector cells that remained bound and do not have the marker associated with synapse formation; or, wherein the functional cellular avidity score is determined by calculating the ratio of the number of target cells associated with said marker associated with synapse formation to the number of target cells that have interacted with the effector cells in the contacting step c) and that did not remain bound with the effector cells after applying the force in step d) and the number of target cells that do not have the marker associated with synapse formation.

In any case, a cellular avidity score is provided herein which allows for a relative measure of cellular avidity which is highly useful, as it allows for a comparison, e.g. in quality control relative to a reference or when comparing different effector cells or the like. It is understood that it is not required to determine the total number(s) of each and every fraction, but that it can suffice to determine the number of cells in a representative portion of a fraction to determine the numbers therein and calculate a score.

In a further embodiment, a method is provided wherein the effector cells that remain bound with the target cells after exerting a force, e.g. at the end of step d), are subsequently collected. Effector cells that remained bound with target cells and attached with the surface can be mechanically collected, e.g. by scraping of the cells from the surface, or by resuspending the cells. This may be done with a suitable suspension buffer or the like and/or by using high shear force flows. The cells thus obtained can comprise doublets and singlets. Hence, in a further embodiment, the effector cells are collected bound to the target cells. Effector cells bound with target cells (as depicted in FIG. 2, (ii) and (iii)) can easily be separated from singlets present, e.g. via cell sorting. Hence, in another embodiment, the effector cells bound to the target cells are separated from the effector cells and/or target cells that did not remain bound after the step of exerting a force (step d).

In a further embodiment, the effector cells that remain bound with the target cells after exerting a force are subjected to a separation step, i.e. the effector cells are detached from the target cells. Accordingly, in this embodiment, effector cells bound with target cells after step d) of applying the force are separated from each other, preferably with trypsin. As shown in the examples, it was found that highly surprisingly, effector cells and target cells that formed a bond, of which a substantial portion is understood to comprise synapses, could be separated from each other with trypsin or the like. Hence, in accordance with this embodiment, the target cells and effector cells that are bound to each other are enzymatically or chemically separated, preferably with trypsin or the like. As shown in FIG. 3, such separation can occur either in situ, i.e. directly on the cells that remained bound to the attached cells after exerting a force (FIG. 3B) or on the cells collected therefrom (FIG. 3A). Either way, single cells can be obtained of target cells and effector cells derived from target cells bound to effector cells that formed e.g. a synapse.

In any case, in the cells obtained, which can be either in the form of target cells bound to effector cells or separated cells, e.g. with trypsin, the marker associated with synapse formation can be determined, which may be in situ or in collected cells. Thus, in a further embodiment, the marker associated with synapse formation is determined in either the target cell or the effector cell. In another embodiment, in the method in accordance with the invention one or more markers associated with synapse formation are determined and the one or more markers are determined in the effector cells and/or in the target cells. When cells are labelled, either in a singlet state or doublet state, cells may highly advantageously be sorted and collected and subsequently analysed.

In one embodiment, a method in accordance with the invention is provided, wherein the marker associated with synapse formation is selected from the group consisting of calcium signalling signatures; spatial clustering of synapse localized molecules such as LFA-1, CD28, CD3, Agrin; changes to internal cell structure and/or cytoskeleton such as F-Actin, Talin, microtubules, centrosome, lytic granules, nucleus position, mitochondrial relocation; changes in effector cell motility; changes in external cell morphology and/or cell shape; and apoptosis of target cells. Synapse formation includes the initiation of a synapse up to and including the establishment of a synapse and subsequent signalling cascade in which, as described above but not necessarily limited thereto, markers associated with synapse formation are associated.

In a further embodiment, the marker for synapse formation is calcium signalling, which can be detected with fluorescent calcium indicators, such as Fura2 AM (available from Invitrogen, item nr. F1221), which marker is suitable for detection in effector cells and in other live cells. Other suitable dyes to detect calcium signalling can be selected from the group of Fura Red AM, Indo-1, pentapotassium, Fluo-3, fluo-4, Calcium Green-1, Rhod-2 and X-Rhod-1, Oregon Green 488 BAPTA. Hence, in one embodiment, the marker for synapse formation is calcium signalling which is detected with an indicator selected from the group consisting of Fura2 AM, Fura Red AM, Indo-1, pentapotassium, Fluo-3, fluo-4, Calcium Green-1, Rhod-2 and X-Rhod-1, and Oregon Green 488 BAPTA. With these markers calcium signalling can be detected which is a hallmark of synapse formation.

Furthermore, membrane potential dyes may also be used as calcium signalling indicators, such as the Invitrogen FluoVolt™ Membrane Potential Kit (Catalog number: F10488). Depolarization of the synapse forming cells using a slow-response potential-sensitive probe such as Invitrogen DiSBAC2(3) (Bis-(1,3-Diethylthiobarbituric Acid) Trimethine Oxonol), Catalog number: B413 may also be used. Hence, in another embodiment the marker for synapse formation is detected utilizing membrane potential dyes.

In another embodiment, the marker for synapse formation is cytoskeleton rearrangement, which can be detected in effector cells with live or fixed cells, using cell staining, e.g. F-actin can be detected with Phalloidin conjugates or CellMask™ (Invitrogen, item nr A57243). Other usable stains can include SiR-Actin, CellLight™ Talin-GFP, BacMam 2.0, or Tubulin Tracker Deep Red. Hence, in one embodiment, the marker for synapse formation is cytoskeleton rearrangement, which is for example detected with a stain selected from the group consisting of Phalloidin conjugates, CellMask™, SiR-Actin, Cell Light™ Talin-GFP, BacMam 2.0, or Tubulin Tracker Deep Red. With these markers, cytoskeleton rearrangement can be detected.

In another embodiment, the marker for synapse formation involves monitoring effector cell motility. Detection of effector cell motility can be performed by video/timelapse monitoring of effector cells that remain in contact with the target cells after the force has been exerted. Detection of synapse formation includes detecting effector cell immobility, i.e. upon synapse formation an effector cell will stop moving and remains into contact with the target cell with which it forms a synapse. Cell motility can be detected by membrane staining of effector cells and imaging, or using, for example, brightfield, darkfield or phase contrast microscopy.

It is understood that for some of the methods for detecting a marker for synapse formation which use e.g. microscopy and monitoring of motility, or short-lived signals, may be combined with having cells provided with e.g. photoactivatable label, to, upon detection of the marker, activate such a label in the cell, such that in subsequent steps, e.g. sorting, FACS analysis or the like, cells for which the marker associated with synapse formation was detected can easily be tracked.

As described above, in accordance with the invention, markers associated with synapse formation can be determined, e.g. via utilizing labels or the like. However, it may be highly advantageous to sequence obtained cells and identify synapse formation by sequencing. It is understood that sequencing comprises nucleotide sequencing, e.g. sequencing of DNA and/or RNA as expressed present in cells. Means and methods are widely known in the art to sequence DNA and/or RNA as expressed in the cell. Hence in another embodiment, in the methods in accordance with the invention the markers are determined with sequencing. This is in particular highly useful for such markers which are up- or downregulated in target cells and/or effector cells in forming a synapse or having a synapse. Suitable markers for T cell activation and synapse formation are transcripts linked to interferon expression, proliferation, and cytokine expression and include: interferon pathway upregulation: CD4, IFIT3, IFIT2, STAT1, MX1, IRF7, ISG15, IFITM3, OAS2, JAK2, SOCS1, TRIM21; proliferation: LIF, IL2, CENPV, NME1, FABP5, ORC6, GOS2, GCK; cytokine expression: CCL3, IFNG, CCL4, XCL1, XCL2, CSF2, IL10, HOPX, TIM3, LAG3, PRF1, TNFRSF9, NKG7, IL26. Sequencing may also be used to identify non-synapse forming cells by detecting molecular signatures linked to resting cell states, such as: FOXP3, CTLA4, MTNFRSF4, IRF4, BATF, TNFRSF18, TOX2, PRDMI, LEF1, ATM, SELL, KLF2, ITGA6, IL7R, CD52, S100A4, TGFB3, AQP3, NLRP3, KLF2, ITGB7.

It may be also advantageous to determine the molecular state of the target cells as this can give an indication of the cell killing potency of the effector cells. In the case of paired analysis where effector-targeT cell doublets are recovered this enables direct linking of effector cell phenotype with their killing capabilities. Next to staining methods for apoptosis commonly used in the field sequencing or qPCR can be used to detect transcripts linked to apoptosis or cell survival in the target cell, markers include: Bcl-2 family (BCL-xL) caspases 3, caspases 7, cleaved PARP, bax, bad, bak, bid, puma noxa, bcl-2, bcl-xl, mcl-1, p53, and cytochrome c, Smac/Diablo, survivn, Mcl-1, RNA Y1.

As it is understood that changes in gene expression may take some time and are not necessarily immediately detectable, one may allow after the step of exerting the force, the cells to remain bound to the target cells for some time and have these remain attached to the surface as well. Alternatively, one may also separate e.g. doublets that remained after exerting the force and subsequently e.g. sort doublets in single wells, and allow these to remain for some time. A suitable time to allow for expression of markers associated with synapse formation may be from 1 hour to 24 hours.

This is highly advantageous as sequencing methods allow for single cell sequencing, which also allows for determining the sequences of receptors expressed by the cell as well. Hence, in a further embodiment, different receptors are determined and identified via sequencing. In yet another embodiment, sequencing comprises single cell sequencing. In still another embodiment, sequencing comprises sequencing the expressed genome. Sequencing the expressed genome is highly useful as it allows to determine up- and downregulated gene expression. Nevertheless, sequencing genomic DNA may be useful as it may also provide useful information e.g. epigenetic markers associated with in vivo potency and durable response.

In a further embodiment, the applied force in the means and methods of the invention is a force ramp, preferably a linear force ramp. It is understood that the force that is to be applied can be a constant force applied for a defined period. The forces applied may be in various forms as a function of time. Preferably however, the applied force is an increasing force, that is, after the incubation step, an increasing force is applied for a defined period until a defined end force is reached. Increasing the force is preferably done as a linear force ramp, but other ways to increase the force over time may also be used (e.g. exponential loading where the force is doubled over a certain small time period and keeps doubling until an end force is reached. For example, as shown in the example section, in about 150 seconds, a defined end force is reached of 1000 pN. An increasing force may be referred to as a force ramp, which may preferably be a linear force ramp, but other ways of increasing the force may be contemplated. As said, detached cells can be collected in a fraction. It may also be opted to collect cells in different fractions, e.g. each fraction representing a defined period in which the force (ramp) was applied and/or a defined force regime. By collecting different detached fractions, one may e.g. construct a cellular avidity curve, i.e. plotting determined fractions at the y-axis, while plotting increasing applied forces at the x-axis.

Any suitable force application method may be contemplated in accordance with the invention. Increasing the force can be well controlled with acceleration-based methods of applying force such as centrifugation, with shear flow and with acoustic force, which are all suitable means to be used in the methods in accordance with the invention but any other means of controllably causing a force on the cells attached to the target cells, thereby forcing them away from the target cells or the functionalized wall surface, may be contemplated. Accordingly, in a further embodiment, the force that is applied is selected from is an acoustic force, a shear flow force or an acceleration force.

It may be advantageous to apply shear flow and/or centrifugal flow in scenarios wherein a large number of cells are to be tested. Acoustic force, though very well suitable in the means and methods of the invention, allows for the processing of about 100-10,000 cells, e.g. utilizing a device like the z-Movi® as available from LUMICKS. This is because the surface area at which the acoustic force may be well controlled can be limited. However, using centrifugal force and/or shear flow, the forces exerted on the cells that attach to the target cell layer can be more easily controlled over much larger areas allowing for the analysing and/or sorting of millions to billions of cells.

With regard to the cellular avidity, it is understood that this is indicative of the strength of binding of cells of interest, i.e. cells carrying a receptor (or control cells thereof) to the target cells. It is understood that where we refer to specific forces applied to cells this may refer to average forces, e.g. such forces may not be fully homogeneous, for example over the contact surface as may be the case with acoustic forces and shear-flow forces (see e.g. Nguyen, A., Brandt, M., Muenker, T. M., & Betz, T. (2021). Lab on a Chip, 21(10), 1929-1947 for a description of force inhomogeneities in acoustic force application).

Also, for shear-flow forces the forces may not be fully homogeneous, for example since the flow speed near the side walls of a flow channel (e.g. with a rectangular cross section) may be lower than in the center of the flow cell (due to the no-slip boundary condition). By choosing a cross section with a high aspect ratio (low and wide), these flow effects may be minimized such that only a few percent of the cells experience a substantially smaller force than the cells in the center of the flow cell. Other methods to mitigate such effects and to specifically select cells that have experienced similar forces may include using flow cell geometries with multiple fluid inlets and/or outlets such that the properties of laminar flow can be used to ensure cells of interest only land in regions of homogeneous force and/or are only selected from regions of homogeneous force. In one example, by using three channel inlets side by side one can use the side channels as sheath flow channels to focus cells of interest inserted into the central channel inlet towards the center of the interaction region where the acoustic and/or shear force may be substantially homogeneous. The sheath flow fluid may be the same buffer fluid as is used for the sample cells but then free of sample cells. By increasing the flow speed through the sheath flow channels, the cells are more focused and confined to the center of the channel, while by reducing the sheath flow speed the cells are allowed to spread out more. Similarly, on the collection side, flows in three side-by-side collection channels may be controlled to possibly discard cells flowing close to the channel boundaries and only collecting cells from the center of the channel. By controlling the relative flow speeds of such side channels and the center channel asymmetrically the location of the effective interaction region of the sorting device can be further controlled and cells that have underwent defined forces can be selected and/or detected.

Further means to enable collection of cells from a specific interaction region (and therefore collection of cells that experienced a defined force) include means and methods wherein cells of interest may be provided with a photoactivatable label which may be subsequently activated by illumination with light of a suitable wavelength only in a well-defined interaction region of the device (e.g. near the center of a flow channel or in a center region under an (acoustic) force transducer) to photoactivate and/or switch the dye. Subsequently, the cells can be sorted for example using fluorescence activated cell sorting (FACS) and only those cells which are activated are further used according to the methods described herein thereby obtaining the cells on which defined forces have been exerted.

For centrifuge forces, it is easier to ensure that the force applied is homogeneous across the whole interaction region, since such a force does not depend strongly on a location on a surface with respect to a force transducer and/or the wall of a flow channel or sample holder.

Accordingly, in connection to the subject matter disclosed herein, means and methods exist which allow one to exert forces on cells attached to a surface and collect the cells, detached and/or attached cells, on which defined forces have been exerted. It is understood that with regard to the force exerted, the exact forces experienced by cells may also depend on cell size and/or other cell properties such as density and compressibility. The force may be a nominal force and not the true force experienced by the cells. E.g. it may be hard to precisely predict the average cell size, density, compressibility, etc. of the cells and the force may have been calculated based on theory alone or may have been calibrated using test particles with specific (preferably known) properties (see e.g. Kamsma, D., Creyghton, R., Sitters, G., Wuite, G. J. L., & Peterman, E. J. G. (2016). Tuning the Music: Acoustic Force Spectroscopy (AFS) 2.0. Methods, 105, 26-33). The force may be such a calculated or calibrated force expressed with units of N (e.g. pN), but it may also be expressed without calibration as the input power (Vpp) applied to a piezo element (see Sitters, G., Kamsma, D., Thalhammer, G., Ritsch-Marte, M., Peterman, E. J. G., & Wuite, G. J. L. (2014). Acoustic force spectroscopy. Nature Methods, 12(1), 47-50), as angular velocity squared (ω²) in the case of centrifugal force application or as flow speed v and or as shear stress (Pa) in applications using shear forces. As long as the forces exerted by the devices, e.g. shear force, acoustic force, or centrifugal forces, but not limited thereto, can be varied and controlled and reproduced in such devices, such devices are suitable for the means and methods in accordance with the invention.

In another embodiment, the target cells are attached to a glass or a plastic surface, preferably a glass surface in a chip or the like (such as described i.a. in Fernández de Larrea, C. et al. (2020). Blood Cancer Discovery, 1(2), 146-154) with target cells attached to its surface. Such a chip is also described in U.S. Pat. No. 10,941,437B2 and WO2018083193). The target cells that are attached to the surface, preferably are attached as a monolayer. The monolayer preferably is at high confluency. The subsequent cells that are to interact with the target cells are preferably provided in a relatively low cell density as compared with the target cells, such that substantially all cells can interact with a target cell (there are more target cells available for the total number of cells comprised in the effector cells). Such provides for advantageous controllable conditions when applying the force on the cells, e.g. on the effector cells.

As said, the target cells may be cancer cells or cells presenting an antigen. With regard to effector cells, immune cells are contemplated in accordance with the invention. In a preferred embodiment immune effector cells are provided as effector cells with a receptor, and target cells expressing an antigen which the cell of interest is to specifically target. For example, effector cells may be selected from T cells, NK cells, dendritic cells, neutrophils, macrophages, or monocytes. Such cells may be genetically modified, e.g. provided with e.g. a CAR. Preferably, effector cells may be CD3+ T cells.

As said, target cells are typically cancer cells, or other cells expressing an antigen, e.g. viral antigens or the like. Hence, in yet another further embodiment, the receptor is a CAR or a TCR.

Further embodiments include methods being performed in the presence of agent(s). For example, one or more agents capable of modulating the cellular avidity between an effector cell with a receptor and a target cell may be included in the steps of contacting effector cells with target cells and subsequently applying a force away from the target cells. Modulating is understood to either enhance or reduce cellular avidity. This way, for example, in case an agent is present that is known to modulate a desired interaction between a cell with a receptor and a target cell, receptor/target interactions may be selected that are not affected by such agents, or, conversely, are aided by such agents. Hence, in another embodiment, an agent capable of modulating the interaction between the effector cells with a receptor and the target cell is included at least in the contacting step and when applying a force. For example, an agent could be used that reduces aspecific binding between a cell with a receptor and target cells.

In another embodiment, a cell engager may be provided. Cell engagers include antibodies, or the like, which are capable of binding to a target cell and an effector cell. Such antibodies may include single chain antibodies comprising two binding domains such as scFv domain, and include BiTEs (i.e. bispecific T cell engagers) or the like. A conventional antibody design includes heavy and light chains, with one half of the antibody (one heavy chain and one light chain) engaging with a target cell, and the other half of the antibody (another heavy chain and another light chain) engaging with an effector cell, wherein preferably, the Fc domain is made inert. In any case, suitable cell engagers are widely known in the art and the current invention allows to study and/or determine cellular avidity, e.g. synapse formation, induced by a cell engager between a target cell and an effector cell. Hence, accordingly, in the means and methods in accordance with the invention, a cell engager may be provided in addition, and e.g. the cellular avidity score is determined, induced by said cell engager between an effector cell and a target cell, said cell engager having a binding region capable of binding the effector cell and a binding region capable of binding the target cell. It is understood that the contacting step in the methods of the invention can thus be performed in the presence of the cell engager to allow the cells to interact, i.e. effector cells and target cells, and form a bond, e.g. a synapse, via the cell engager.

Accordingly, in one embodiment, in methods in accordance with the invention, a cell engager is provided capable of binding the effector cell and the target cell and inducing synapse formation, and the cell engager is included in the contacting/interacting step. Furthermore, such methods are highly useful for screening cell engagers, e.g. by identifying cell engagers that are particular capable of inducing a synapse.

It was observed that when cells that remained bound and attached after a cellular avidity measurement were resuspended utilizing i.a. repeated pipetting and thus exerting a significant force, such as described e.g. in the example section, cells attached to the surface were detached and moreover, a portion of the cells that were bound to the attached cells became unbound. Hence, this implied that with such a process step a differential force can be applied on bound cells that can exceed the maximum force that is applied away from cells attached to a surface. This way, further cell-cell bonds that may be aspecific cell-cell bonds may thus be broken therewith providing substantially effector cells bound with target cells (and optionally a cell engager) that formed a synapse can be retained. Hence, in a further embodiment, after the force has been applied away from the attached cells, subsequently, the cells are resuspended and a differential force is applied such that substantially cells that are bound to each other via a synapse remain bound to each other and substantially cells that have formed aspecific bonds are unbound, i.e. have their cell-cell bonds broken. It is also understood that instead of applying the force away, the differential force may be applied instead, and that by applying a differential force, it may no longer be required to have cells attached to a surface, i.e. immobilized. Furthermore, it is also understood that after the differential force has been applied, cells are highly preferably separated and/or sorted in order to avoid further interaction between cells to avoid establishing additional cell-cell bonds. Hence, highly preferably, cells are collected and sorted and/or analysed after the differential force has been applied.

As is clear from the above, the type of force that is to be applied in accordance with the invention is a force capable of breaking cell-cell bonds, i.e. the force exerted causes cells bound to each other to move away from each other to such an extent that a cell-cell bond may break or rupture. A differential force means that the force on one cell differs from the force on the other cell with regard to direction of the force and/or the magnitude of the force, resulting in a net force allowing to break cell-cell bonds if the differential force exceeds the binding force.

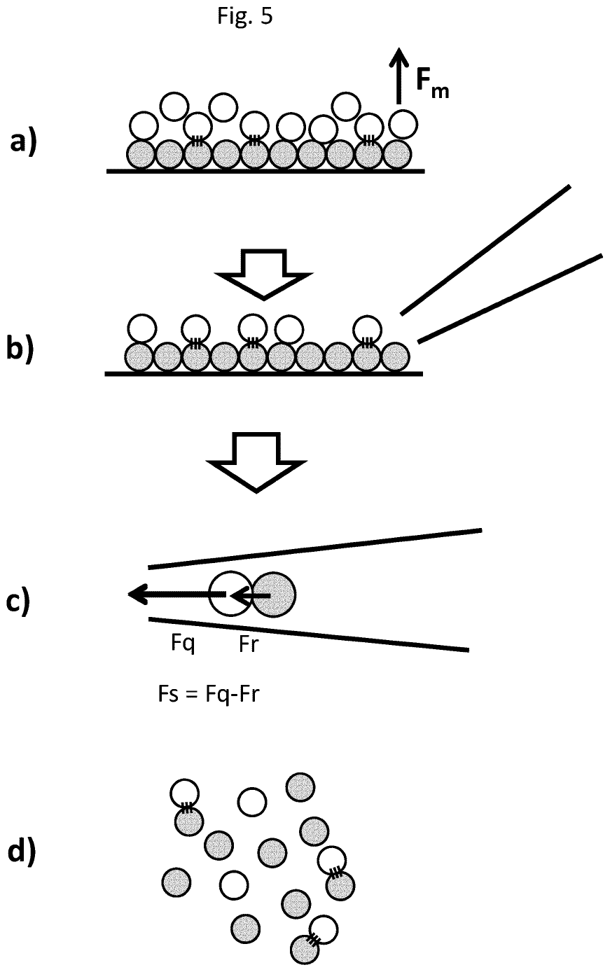

For example, when a target cell bound to an effector cell, a doublet, is forced through a nozzle, the closer to the throat of the nozzle, the faster the flow. This means that the first cell to enter the nozzle is subjected to a stronger acceleration than the cell lagging behind and the cells experience a differential force resulting in a net force which can result in cell-cell bond rupture, provided the force is large enough (see e.g. FIG. 5, in particular 5c). A differential force that can be applied includes a shear force, e.g. such as can be applied utilizing repeated pipetting (repeated upwards and downwards flow of the sample) or flow through a nozzle.

Other means and methods are known in the art with which shear forces can be applied to cells, e.g. flowing a cell suspension at a constant speed and bombarding these cells to a flat surface at a defined angle. Furthermore, forcing a cell suspension through a needle with a defined internal diameter and a defined force may provide for a well controllable shear force as well. The cell suspension may be subjected to several rounds of such process steps to ensure substantially all cell-cell bonds experience the maximum force that may be achieved with the process step. Such a process step allows for automation, enabling control and repeatability of the process, therewith controlling shear forces exerted. Suitable devices for breaking apart cell-cell bonds which are not synapses are known in the art (e.g. Zahniser et al., J Histochem Cytochem. 1979, 27(1), 635-641). Also, by properly tuning the forces in a flow-cytometer normally used to measure cell deformations, such as e.g. described in Otto, et al. Nat Methods 12, 199-202(2015), suitable forces can be applied. Tuning can be achieved e.g. by changing the nozzle size or geometry and/or the flow speeds used.

Other suitable devices known in the art may include for a vortex mixer, with which shear forces may be suitably applied as well. Accordingly, in one embodiment, the force applied involves a shear force.

In another embodiment, the force applied is an ultrasonic force. It is understood, as outlined above, that such ultrasonic forces are not forces such as applied e.g. in a device as available from LUMICKS, wherein the force is away from attached cells (e.g. such as in the LUMICKS z-Movi® Cell Avidity Analyzer, e.g. as used by Larson et al., Nature 604, 7906:1-8, Apr. 13, 2022). It is also understood that the ultrasonic force is selected such that cells are not lysed. Hence, appropriate ultrasonic forces can be applied to cells such that cell-cell bonds can be ruptured, which more preferably includes breaking aspecific cell-cell bonds and less preferably breaks specific cell-cell bonds in which an immune synapse is formed. Examples of using ultrasonic forces to break (aspecific) cell-cell bonds, are known in the art (e.g. as described in Buddy et al., Biomaterials Science: An Introduction to Materials in Medicine, 3^{rd} edition, 2013, Chapter 11.2.8, page 576; and Moore et al., Experimental Cell Research, Volume 65, Issue 1, 1971, Pages 228-232).

In any case, suitable applied forces which are known in the art include e.g. a force in the range of 50 pN-10 nN, which said force is a net force exerted on one cell relative to the other cell, of two cells bound to each other. Which means the force is exerted on the cell-cell bond. In another embodiment, the force exerted on one of the two cells relative to the other cell is at least 50 pN, or at least 100 pN, or at least least 200 pN. In another embodiment, the force exerted is at most 10 nN, at most 5 nN, at most 3 nM, at most 2 nM, or at most 1 nN. In yet another embodiment, the force is selected from the range of 1 pN-10 nN, from 100 pN-10 nN, from 500 pN-10 nN, from 1 nN-10 nN. In still a further embodiment, the force is selected from the range of 500 pN-5 nN, from 500 pN-4 pN, from 500 pN-3 pN. For example, a suitable amount of force that can be exerted between cells (e.g. such as in the z-Movi® device) can be selected to be in the range of 200 pN-3000 pN. Of course, these force ranges are known to be useful with cells attached to a surface, and the maximum force that may be selected may exceed 3000 pN as it is not required to have the cells attached to a surface in accordance with the invention.

Accordingly, it is understood that in these embodiments, the differential force to be applied does not require either of the target cells or effector cells to be attached, and the differential force is a force selected from the range of 50 pN-10 nN. In another embodiment, in methods in accordance with the invention wherein the force that is applied is a differential force, neither the target cells nor the effector cells require to be attached to a surface, and the differential force is applied in the range of 50 pN-10 nN, thereby providing cells substantially comprised of target cells, effector cells, and effector cells bound with target cells via a synapse.

Without being bound by theory, as is understood the range of force that may break an aspecific cell-cell bond versus a specific cell-cell bond that forms a synapse differs. This difference can be to such an extent that the ranges of the required forces do not overlap. It is understood that some overlap may occur. Hence the force that is selected, as outlined above, may allow for aspecific cell-cell bonds remaining and some specific cell-cell bonds that formed a synapse to break. In case there is substantially no overlap, a differential force can be selected, as outlined above, which allows substantially for aspecific cell-cell bonds to break, while substantially retaining specific cell-cell bonds that formed a synapse. In case there is no overlap, and ranges are sufficiently far apart, a differential force may be selected, as outline above, which allows for aspecific cell-cell bonds to break while retaining specific cell-cell bonds that formed a synapse. As also outlined herein, the portion of cell-cell bonds that remains after exerting a force and has a synapse can be determined by determining the presence or absence of a marker associated with synapse formation.

In one embodiment, after the step of applying a force in the methods of the invention, the cells are resuspended and a subsequent differential force is applied such that formed aspecific cell-cell bonds are broken. In another embodiment, in methods of the invention wherein either the target cells or effector cells (carrying a receptor) are attached to a surface, this attachment is optional, and, in the step of applying the force instead a differential force is applied. In yet another embodiment, in methods of the invention wherein either the target cells or effector cells are attached to a surface, this attachment is optional, and, in the step of applying the force instead a differential force is applied, wherein the differential force is such that cells that are bound via a synapse remain bound to each other and formed aspecific cell-cell bonds are broken.

In a further embodiment, a method is provided for determining the presence or absence of a marker associated with synapse formation, comprising the steps of:
a) providing effector cells carrying a receptor;
b) providing target cells;
wherein the effector cell carrying a receptor is capable of binding target cells, and wherein the effector cells or the target cells are attached to a surface;
c) contacting the effector cells with the target cells to allow the cells to interact with each other;
d) applying a force away from the cells attached to the surface, such that at least part of the cells bound to the cells attached to the surface move away therefrom;
e) detecting for effector cells that have remained bound with the target cells and attached to the surface after applying the force the presence or absence of a marker associated with synapse formation.

Hence, in the first step of the method, effector cells carrying a receptor are provided. In accordance with the invention, effector cells carrying a receptor include effector cells of the immune system that can exert an effect, via the receptor. For example, a T cell carrying a T cell receptor can bind an antigen on a cancer cell, upon which it can e.g. exert a cytotoxic effect and kill the target cell. Effector cells can be derived from nature, e.g. obtained from a host, and can also include genetically modified cells wherein e.g. a receptor in particular useful is provided to an effector cell.

Next, target cells are provided. Target cells in accordance with the invention are the cells on which the effector cells are to exert an effect, e.g. bind therewith and trigger an immune reaction thereto. Target cells include cancer cells presenting an antigen. An antigen may be presented by MHC, i.e. HLA in humans, which are specialized receptors that present peptides e.g. derived from digested proteins expressed by the cell (e.g. usually 8-11 amino acids in length for MHCI). An antigen may also be a protein or other biomolecule that is presented on the surface of a cell, e.g. epidermal growth factor receptors or checkpoint proteins, which in the case of cancer cells are overexpressed therewith providing a differentiating feature. Target cells may also include cells expressing auto-antigens, e.g. known to be involved in autoimmunity diseases or cells infected with a pathogen, e.g. a virus.

Subsequently, either the target cells or the effector cells are attached to a surface. It is understood that surfaces for attaching cells may be any surface suitable for attaching cells. Suitable surfaces for attaching cells include plastic or glass surfaces. These surfaces may be coated e.g. with a protein to attach cells to the surface, such as poly-L-lysine or the like. The attachment of the cells to the surface is such that the strength of the binding to the surface is sufficient to have the cells remaining attached when applying a suitable force (see i.a. FIG. 1).

The effector cell carrying the receptor is capable of binding target cells, or the effector cell may be analysed for its capability of binding target cells, e.g. in screening and discovery methods as envisioned herein. It is understood this capability of binding target cells includes a specific interaction that can induce synapse formation, i.e. the effector cell is to bind to a target cell and exert an effect thereon, e.g. induce target cell killing.

In the next step, effector cells and the target cells are contacted with each other to allow the cells to interact. This step is such that the effector cells will have sufficient time to interact with target cells and can form a bond, including synapses (see i.a. FIG. 2). It is understood that a cell-cell interaction may not always result in a cell-cell bond, which can be a synapse or aspecific bond. The contacting step is such that cell-cell bonds can be formed and appropriate conditions therefore are selected. It is understood that because the conditions are selected such that a synapse can be formed, this necessarily implies that aspecific cell-cell bonds are allowed to be formed at the same time.

Once the effector cells have had contact with the target cells, and have had the opportunity, if possible, to form a synapse, in a subsequent step a force is applied away from the cells attached to the surface, such that at least part of the cells bound to the cells attached to the surface, and unbound cells as well, move away from the cells attached to the surface. This way, target cells are obtained with effector cells bound thereto, which are attached to the surface, wherein the exerted force was not sufficient to break cell-cell bonds. Of course, cells that are attached to the surface, to which no subsequent cells are bound, remain as well.

As said, the force is applied in a direction away from the attached cells. It is understood that in this step, the force applied may be perpendicular (in the direction of z-axis) to the surface (x,y) to which cells are attached, for example when a centrifugal force or acoustic force is applied. The force may also be lateral (in the direction of the x-axis or y-axis relative to the surface), for example when a shear force is applied. In any case, the force is applied and is controlled such that a defined force is exerted on the cells that interacted with the attached cells. It is understood that the force that is exerted on the cells interacting with attached cells is to be substantially equal, such can be achieved e.g. when using a flat surface. Other suitable surface shapes may be used (e.g. a tube with exerted concentrical force or laminar flow force in the direction of the length of the tube), as long as the force exerted can be substantially equal at a defined surface area to which cells are attached, such a surface shape may be contemplated.

The cells that are attached are preferably attached to a glass or plastic surface, preferably a surface in a chip, which allows for detection of cells e.g. via microscopy or other means. The applied force required to move a cell away from an attached cell preferably can be detected, e.g. via microscopy or other means, to which may be referred to as a cell detachment event or cells moving away. This way, cells moving away can be monitored and counted. It may be advantageous and convenient to use microscopy, with which bound cells can be identified and quantified and cells moving away can be likewise monitored and quantified, also allowing for detection of markers. For example, the z-Movi® device as available from LUMICKS which applies an acoustic force may be well equipped to do so. Likewise, similar devices may be provided with microscopy or other means to quantify cells, detect markers and bound cells and/or cells moving away, and also utilizing e.g. shear force or centrifugal forces instead of acoustic force.

The effector cells that have remained bound with the target cells and attached to the surface have subsequently detected therein or associated therewith the presence or absence of a marker associated with synapse formation. It is understood that with regard to the effector cells that have remained bound with target cells and attached to the surface, and that have a marker associated with synapse formation determined, means that either the determined marker is from the effector cell or from the target cell. It is also understood that one or more markers may be determined. It may also be contemplated that one or more markers are determined of the effector cell and one or more markers are determined of the target cells. As long as the one or more markers associated with synapse formation that are determined can be assigned to an effector cell bound to a target cell, such a marker determination can be contemplated. Suitable means and methods to determine markers in an effector cell or target cell are described herein further below.

It is understood that a synapse is a specialized structure that forms when the plasma membranes of two cells come into close proximity to transmit signals. Cells of the immune system form synapses that are essential for cell activation and function. Lymphocytes such as T cells, B cells and natural killer (NK) cells form synapses that can be referred to as immunological synapses. Such a synapse typically forms between immune effector cells and target cells, e.g. cells presenting an antigen. A non-limiting example is e.g. a T cell or a CAR-T cell and a cancer cell. The formation of synapses between e.g. an effector cell and a target cell, for example an APC (antigen presenting cell), is a hall-mark event and signals the presence of specific interactions (i.e. the specific interaction between, for example, a TCR or CAR and an antigen recognized thereby) between the effector cell and the target cell that are involved in the formation of such immunological synapses.

In the case of a T cell, a synapse can be formed between the lymphocyte and antigen-presenting cells (APCs) during the recognition of the peptide antigen-major histocompatibility complex (pMHC) ligand by the T cell antigen receptor (TCR). The TCR and pMHC are both membrane-bound, so the TCR will only be triggered by its ligand at the interface between T cells and APCs. A synapse can be observed at the T cell-APC interface as concentric rings by confocal microscopy, often referred to as “bull's eye” (Huppa, J. B., & Davis, M. M. (2003). T cell-antigen recognition and the immunological synapse. Nature Reviews Immunology, 3(12), 973-983). These rings were named the central, peripheral, and distal supramolecular activation cluster (i.e. respectively cSMAC, pSMAC and dSMAC). The TCR has been reported to be present in the cSMAC, whereas other lymphocyte specific proteins such as lymphocyte function-associated antigen-1 (LFA-1), are integrated into the pSMAC ring that surrounds the TCR. The formation of this ringed structure (“bull's eye”) is however not universal and other formations such as “multifocal immunological synapses” between T cells and dendritic cells, or the like, have been described.

The immunological synapse can be considered to be any structure formed at the interface resulting from a functional and specific effector-target cell interaction, such as for example T cell-APC contacts. Markers associated with effector cells and synapse formation include one or more of CD43, CD44, CD45, LFA-1, Talin, F-actin, ZAP70, CD2, CD4, CD8, CD3, CD28, PD-1, ICOS, and TCR. Markers of target cells include one or more of ICAM-1 (associates with LFA-1), CD48/58 (interacting with CD2) CD80/CD86 (interacting with CTLA-4 and CD28), PDL1/PDL2 (associating with PD-1), and MHC presenting the antigen (that specifically interacts with the TCR). These markers, and concentrations thereof, may be detected e.g. with fluorescent labels at the interface between effector cell and target cell, or intracellularly in close proximity to the synaptic interface.

For example, markers of effector cells that have been associated with dSMAC are CD43, CD44, CD45. The effector cell markers LFA-1, Talin, F-actin, CD2, CD4 and CD8 have been associated with pSMAC. The markers CD3, CD28, PD-1, ICOS, and TCR of effector cells have been associated with cSMAC. Markers that have been associated with a synapse on a target cell are ICAM-1 (associates with LFA-1), CD48/58 (interacting with CD2) CD80/CD86 (interacting with CTLA-4 and CD28), and PDL1/PDL2 (associating with PD-1), and of course an MHC presenting the antigen (that specifically interacts with the TCR). These markers have been shown to be associated with synapses in a TCR-MHC interaction. Likewise, cell engagers (such as a bispecific antibody that e.g. binds CD3 on an effector cell with one arm and with the other arm an antigen on a target cell) which engage an effector cell with a target cell, can trigger the formation of a similar synapse structure as observed with a classic TCR-MHC interaction.

With regard to CARs of e.g. CAR T cells, these are chimeric antigen receptors that are to mimic a TCR or the like. CARs are engineered. The first generation of CAR were provided with an antigen recognition part often an antibody derived region (e.g. a scFv) fused to a transmembrane region and intracellular region of e.g. a CD3 ζ-chain. Later generations combined intracellular signalling domains from various costimulatory protein receptors (e.g. CD28, 41BB, ICOS) incorporated in the cytoplasmic tail of the CAR to enhance further signalling. Further generations also incorporated in their design an inducible release of transgenic immune modifiers, such as IL-12, to shape the tumor environment by augmenting e.g. T cell activation, attracting and activating innate immunity. As CARs often have antibody variable regions incorporated, these can target e.g. receptors themselves that are presented at the surface of a cell (e.g. Her2, PD-1 etc.), or can also target antigens presented by MHC, derived e.g. from proteins intracellular processed by the ubiquitin-proteasome system. Such peptides presented by MHC include proteins that are processed internally and presented by MHC, which can be derived from receptors, secreted proteins, intracellular proteins or internalized proteins. Synapses formed between CARs and target cells may not provide a classical bull's-eye like structure with a well-characterized SMAC domain, but may result in less organized patterns. Multiple CAR microclusters form and signalling molecules associate, which are dispersed in the center of the synapse interface.

Synapse formation is a spatiotemporal process that starts e.g. by TCR binding to MHC or binding of an antigen with CAR or cell engagement, and subsequent phosphorylation of the cytosolic tails of CD3 resulting in a triggered state. This sets of a cascade of processes that result in an activated T cell state, which also depends on the effector cell type and its phenotypic state. Key processes include: calcium signalling, internal cell structure and/or cytoskeleton changes of effector cells, involving F-Actin, Talin, and changes in microtubules, centrosomes, lytic granules, nucleus position, and mitochondrial location. Transactivation of adhesion molecules, cytokine and marker expression. IFNγ, granzyme and perforin may be released by effector cells to thereby induce i.a. target cell killing. Also, in addition, in target cells apoptotic markers can be found, including ICAM-1 clustering, phosphatidyl translocation, mitochondrial depolarization, caspase-3 activation and DNA fragmentation. Hence, various stages of specific effector cell and target cell interaction and immune activation can be detected.

In any case, synapse formation, or a synapse can be determined by staining, i.e. with fluorescent labels, ligands, antibodies, or probes, or the like, which may be used on live cells or on fixed cells targeting the mechanisms involved in T cell activation and/or synapse formation. Sequencing based methods may also be used to identify activated T cells and thereby associate mRNA levels with the formation of stable synapses. T cell activation leads to changes in mRNA stability and expression. E.g. increases in expression of cytokine or secretory transcripts (IL2, IFNγ, granzyme, perforin) and proliferation pathways and either bulk or single-cell RNA sequencing may be used to detect these changes and correlate these to the number or synapses formed. Hence, synapse formation and effector cell activation, including its stages, can be detected by means of staining, fluorescent labelling, sequencing and the like.

For example, as indicated below and as described in the examples herein, an effector cell-target cell pair that remains bound after the force has been exerted, may be isolated and subsequently stained or sequenced to detect synapse formation. The cells may be optionally fixated, e.g. with methanol, glutaraldehyde or para-formaldehyde or the like.

Hence, of the effector cells that have remained bound with target cells and attached to the surface, a marker associated with synapse formation, can thus be determined. As a synapse is formed between effector cells and target cells, the number of effector cells or target cells associated with said marker each represents the number of synapses formed, hence, determining either may suffice. Off course, one may also determine a marker for both effector cells and target cells.

As said, either the effector cells or the target cells may be attached to a surface. It may be preferred, in one embodiment, to have the target cells attached to a surface. This is because e.g. target cells may be more easily attached to a suitable surface.

In any case, as outlined above, means and methods for detecting the presence or absence of a marker associated with synapse formation are provided herein. Such means and methods are highly useful and have applications in methods of screening and discovery as well.

For example, in one embodiment, a further method is provided for identifying a receptor capable of binding target cells, from a heterogeneous cell population comprising a plurality of cell populations carrying a variety of receptors, comprising the steps of:
a) providing a heterogeneous cell population comprising a plurality of cells carrying a variety of receptors;
b) providing target cells attached to a surface;
c) contacting the plurality of cells carrying the receptor with the target cells to allow the cells to interact with each other;
d) applying a force on the plurality of cells carrying a variety of receptors away from the target cells, such that at least part of the plurality of cells carrying a variety of receptors detach and/or move away from the target cells;
e) detecting one or more cells carrying a receptor that remain attached to the target cells having a marker associated with synapse formation and determine from said one or more cells with said marker the receptor thereby identifying a receptor capable of binding target cells.

In this embodiment, instead of providing cells expressing a single receptor, a plurality of cell populations, such as effector cells, are provided expressing different receptors, hence a variety of receptors. Highly preferably, such cells expressing a receptor are effector cells, such as T cells or the like. It is understood that these cells represent a heterogeneous cell population, e.g. such as provided with e.g. primary T cells obtained from a donor or patient. Alternatively, a heterogeneous cell population may be generated from e.g. a library of receptors. Each receptor with a defined sequence, e.g. a defined CAR sequence or defined alpha and beta chains of a TCR, or the like, may be represented by a population comprising one or more cells, such as effector cells, to which these receptors were provided (e.g. via transfection or transduction). It is understood that by detecting a cell carrying a receptor attached to target cells, and a marker associated with synapse formation, and determining the receptor thereof, receptors capable of binding target cells are identified that in particular are highly useful as these are capable of inducing synapse formation as well.

For example, a library of CAR-T cells may be prepared from a lentiviral vector library encoding 100 different CAR sequences. With this vector library, 100,000 cells may be transduced. In accordance with the invention, transduced cells carrying the same CAR sequence may be referred to as a population, which population size is on average 1000 cells, and the number of cell populations is 100. Of course, the transduced cells may be grown or expanded to 1,000,000 cells, which increases the average size of a cell population to 10,000, whereas the number of cell populations, e.g. cells carrying a the same receptor (construct), remains 100. Likewise, the same applies to e.g. primary cell samples, in which e.g. clonal expansion may have occurred e.g. in case of T cells or the like. However, although detecting more than one of the same receptor sequence may be useful, as it may provide for further validation of identified receptor sequences, it is not required to detect more than one.

Hence, the current invention allows to identify from heterogeneous cell populations receptors, e.g. via sequencing, as well as markers associated with a synapse. In case a receptor is identified along with a marker associated with synapse formation, this advantageously identifies a receptor sequence which is capable of inducing synapse formation. One may also quantify the number of cells having a marker for synapse formation and the receptor. In case e.g. the number of each population comprised in the heterogenous cell population is similar, one can simply rank the number of each uniquely identified receptor sequence which is identified along with a synapse marker. It is understood that identifying a receptor may include e.g. different (fluorescent) markers associated with receptors and/or can include sequencing methods with which receptor sequences are identified, e.g. by sequencing the variable domains of a receptor.

One may also determine cellular avidity scores for identified receptor sequences that are associated with a synapse marker. For example, the number of cells presenting each receptor may be determined (or provided, or a fraction thereof), e.g. of the cells initially provided, and a ratio calculated of the number of cells with the receptor and the synapse marker to the number of cells initially provided. This way, cellular avidity scores can be determined based on synapse formation, from heterogeneous cell populations, without the need to resort to isolating individual cells (prior to force application), or cloning of TCR receptors or the like, and determining cellular avidity scores for separate receptors or separate effector cells. The more cells detected in fraction(s), such as bound and/or unbound fractions (i.e. attached and/or detached), the more confidence can be obtained with regard to cellular avidity scores, and/or relative cellular avidity, therewith validating further identified receptor sequences.

In any case, by identifying a receptor sequence and a marker associated with synapse formation from a cell with a receptor bound to a target cell, receptor sequences are identified that are highly useful, e.g. as candidate receptors for cell therapy. This allows to identify useful sequences e.g. from human patients or from (artificial) libraries of receptor sequences.

Hence, in a further embodiment, the method further comprises the step of selecting identified receptor sequences and manufacturing a vector comprising such sequences for expressing the receptor in a suitable host cell. Suitable vectors are well known in the art and include e.g. retroviral vectors and lentiviral vectors. Subsequently, the vector is used to transduce or transfect an effector cell to thereby provide for effector cells with the identified receptor. For example, primary cells may be obtained from a patient or a donor which can be subsequently transduced or transfected with the manufactured vector. The subsequently prepared cells may subsequently be transferred to the patient. Hence, the current invention also provides for vectors, such as viral vectors, obtainable by the methods as described herein, as well as cells transduced therewith, obtainable by the methods as described herein. Moreover, the current invention also provides for cells provided with identified receptors, as obtainable by the means and methods as described herein.

Further to the marker associated with synapse formation, the presence of further phenotypic markers can be detected which may add further useful information related to cellular avidity or cellular avidity scores of subpopulations of cells defined by phenotypic markers (Rossi et al. (2018) Blood, 132(8), 804-814; Fraietta et al. (2018) Nature Medicine, 24(5), 563-571). For example, in a scenario wherein e.g. different donors would be used, cell clone subpopulations for each donor may be identified using marker genes as it has been shown that best clinical outcomes are associated with polyfunctional or memory like T cell subsets. Or, when for example different types of cells are provided or when there would be heterogeneity of the provided cells, e.g. when donor cells are used and provided with a receptor of choice. Different donors and/or different types of cells may be labelled differently and/or identified e.g. based on intrinsic genetic differences between the cell types or the like. This can provide in addition to cellular avidity per se, a further complexity to the analysis by which cellular avidities can be determined and compared across cell receptors, and/or across different cell types and/or different donors. Non-limiting examples of suitable phenotypic markers are CD4, CD8, IL-6, STAT3, CD27 and the expression of combinations of cytokines and chemokines known to be associated with durable in vivo responses including: IL17A, granzyme B, IFN-g, macrophage inflammatory protein MIP-1a, perforin, tumor necrosis factor TNF-a, TNF-b, GM-CSF, IL-2, IL-5, IL-7, IL-8, IL-9, IL-12, IL-15, IL-21, IL-4, IL-10, IL-13, IL-22, TGF-b1, sCD137, sCD40L, CCL-11, IP-10, MIP-1b, RANTES, IL-1B, IL-6, IL-17F, MCP-1, MCP-4.

Identifying such further phenotypic markers may be useful, for example when heterogeneous cell populations are provided, and it is of interest to determine the phenotype of a cell that formed a synapse, in addition to identifying the receptor sequence. This way, particular useful phenotypes may be identified which may be of use. For example, if a particular phenotype of an effector cell with a defined receptor is identified which in particular is capable of forming a synapse with a target cell, in a subsequent therapeutic setting, such phenotypes of effector cells may be selected for cell therapy or culture conditions may be selected favourable for such phenotypes. Hence, in a further embodiment, a method is provided for identifying an effector cell with a receptor capable of binding target cells, and the phenotype of the effector cell is identified as well, from a heterogeneous cell population comprising a plurality of cell populations of effector cells carrying a variety of receptors, comprising the steps of:
a) providing a heterogeneous cell population comprising a plurality of cells carrying a variety of receptors;
b) providing target cells attached to a surface;
c) contacting the plurality of cells carrying the receptor with the target cells to allow the cells to interact with each other;
d) applying a force on the plurality of cells carrying a variety of receptors away from the target cells, such that at least part of the plurality of cells carrying a variety of receptors detach and/or move away from the target cells;
e) detecting one or more effector cells carrying a receptor that remain attached to the target cells, having a marker associated with synapse formation, and determining from said one or more effector cells associated with said marker the receptor thereby identifying a receptor capable of binding target cells and furthermore determining the phenotype of said one or more effector cells associated with said marker.

This way, effector cell phenotypes and receptor sequences combined can be identified that are capable of forming a synapse with a target cell, when provided to effector cells with defined phenotypes.

Identifying such further phenotypic markers may also be useful for example, when a heterogeneous cell population is provided with a defined receptor, e.g. a CAR, or a heterogeneous cell population comprises a defined receptor, e.g. as it is derived from a cell clone, and the cell heterogeneous cell population comprises a variety of phenotypes, e.g. because cells have underwent differentiation (in case of derived from a cell clone) or because these cells were generated from primary cells e.g. by transducing such cells with a vector expressing a single receptor. From such heterogeneous cell population it may be useful to identify effector cell subtypes that with a defined receptor are in particular suitable for binding with target cells and forming a synapse. Hence, in another embodiment, a method is provided for determining the phenotype of an effector cell carrying a receptor from a heterogeneous cell population carrying the receptor, comprising the steps of:
a) providing a heterogeneous cell population of effector cells comprising a receptor;
b) providing target cells attached to a surface;
c) contacting the heterogeneous cell population with the target cells to allow the cells to interact with each other;
d) applying a force on the heterogeneous cell population away from the target cells, such that at least part of the cells of the heterogeneous cell population detach and/or move away from the target cells;
e) detecting one or more effector cells carrying the receptor that remain attached to the target cells, having a marker associated with synapse formation, and determining the phenotype of said one or more cells, associated with said marker, thereby identifying phenotypes of effector cells which when provided with said receptor are capable of binding target cells.

This way, effector cell phenotypes can be identified that, when provided with a defined receptor, are capable of binding target cells, and form a synapse with the target cell. Such is highly useful for example in selecting e.g. suitable subpopulations of effector cells for cell therapy.

It is understood that in some embodiments as described above, target cells were attached to the surface. This may be preferred because the cells that bind with the target cells and which are of interest, e.g. because the method is aimed at identifying their receptors and/or phenotype as well as their capacity to induce synapse formation, can be easily be separated from non-binding cells or aspecifically binding cells by applying a force (see e.g. FIGS. 1 and 2). However, these methods may also be employed with having the cells of interest, e.g. cells carrying a receptor or effector cells, attached to the surface. This may make subsequent analysis and/or sorting of the cells of interest perhaps more cumbersome, as said, because it is advantageous that effector cells that do not remain bound or bind to the target cells can be simply removed, but that does not mean that such reverse scenarios may not be contemplated, and may have advantageous for other reasons as well. For example, in scenarios wherein markers associated with synapse formation are to be identified in target cells. As said, detecting effector cells (carrying a receptor) that remain attached to the target cells after exerting a force, having a marker associated with synapse formation, includes determining a marker in target cells or effector cells. Having effector cells attached to the surface can allow for convenient and more efficient detection of markers, and discovery thereof as well, associated with synapse formation in target cells.

Moreover, the methods of the invention may also be utilized to identify novel markers associated with synapse formation. Hence, in another embodiment, a method is provided for identifying a marker associated with cellular avidity, e.g. synapse formation, of an effector cell carrying a receptor which is capable of binding target cells, comprising the steps of:
a) providing effector cells carrying a receptor;
b) providing target cells;
wherein the effector cells carrying the receptor are attached to a surface;
c) contacting the effector cells carrying the receptor with the target cells to allow the cells to interact with each other;
d) applying a force away from the target cells attached to the surface such that at least part of the effector cells bound to target the cells attached to the surface detach and/or move away therefrom;
e) detecting effector cells carrying the receptor bound to the target cells that remain after step d) and identifying the presence or absence of markers.

In another embodiment, a method is provided for identifying a marker associated with cellular avidity, e.g. synapse formation, of an effector cell which is capable of binding target cells, comprising the steps of:
a) providing effector cells;
b) providing target cells;
wherein the effector cells or the target cells are attached to a surface;
c) contacting the effector cells with the target cells to allow the cells to interact with each other;
d) applying a force away from the cells attached to the surface such that at least part of the cells bound to the cells attached to the surface detach and/or move away therefrom;
e) detecting effector cells bound to the target cells that remain after step d) and identifying the presence or absence of markers.

As outlined above, the means and methods as outlined herein are highly useful for detecting markers associated with synapse formation. However, the steps of the method as described herein are also highly useful for identifying novel markers associated with cellular avidity, and, for example synapse formation. This is because after a force has been applied, resulting in substantially the cell-cell bonds to remain that formed a synapse, novel markers can be identified in the effector cells carrying the receptor bound to target cells, and in the target cells as well. For example, by a proteomics approach or analysis of the expressed genome, and comparing the presence or absence of markers (e.g. proteins or expressed genes) e.g. with effector cells and/or target cells that were not allowed to interact with each other, markers can be identified that are associated with synapse formation. It is understood that with regard to the presence or absence of a marker, this may be defined relative to threshold level, e.g. of an appropriate control. Moreover, the identified presence or absence of markers may also be compared with the presence of a marker associated with synapse formation, i.e. known markers associated with synapse formation. Hence, in one embodiment, in the detection step, e), the presence of a marker associated with synapse formation is determined as well and compared with the identified presence or absence of markers. Hence, when markers are identified, and the presence or absence thereof correlates with a known marker for synapse formation, this way, novel markers associated with synapse formation can be identified. Furthermore, the number of cells having an identified marker may be determined as well, and a cellular avidity score can be assigned to an identified marker. When the identified novel marker has a highly similar cellular avidity score as compared with a known marker associated with cellular avidity, such an identified novel marker can be determined to be a marker associated with synapse formation as well.

It is understood that when reference is made to a cellular avidity score, this is to reflect the number of cells that remain bound after the force has been exerted, e.g. in case effector cells, with a receptor, are bound to target cells attached to a surface, the number of cells that remain bound relates to cellular avidity. A cellular avidity score takes into account the (relative) number that remained bound. Of course, as in the means and methods of the invention, markers can be determined as well associated with synapse formation, the number of target cells and/or effector cells that remain bound and have a marker associated with synapse formation can be used as a measure of cellular avidity. In any case, based on the determined number of effector cells and/or target cells associated with marker, a cellular avidity score can be determined. The higher the number of cells associated with said marker, the higher the cellular avidity score. Cellular avidity can be expressed in different ways taking into account the number of cells that have formed a synapse, relative to the number of cells that interacted with the attached cells, or relative to the number of cells that remained bound after exerting a force, or the number of cells that did not remain bound after exerting a force and the number of cells that bound but did not form a synapse. For example, a cellular avidity score can be determined by calculating the ratio of the number of cells associated with said marker to the number of cells contacted or the number of cells not associated with the marker. As depicted in FIGS. 2 and 3, different fractions at different steps of the methods can be collected and the number of cells as determined from such fractions or known (e.g. of a starting fraction) can be used to determine a cellular avidity score in accordance with the invention, taking into account the number of cells associated with synapse formation (e.g. fraction (iii) in FIG. 2). Of course, cellular avidity scores may also be determined in situ by detecting bound cells to the attached cells, which may include detecting markers associated with synapse formation as well (e.g. of bound cells such as depicted in FIG. 2d). In any case, determining a cellular avidity score requires at least the number of cells that remains bound or the number of cells that remain bound and have a marker associated with synapse formation.

With regard to the cellular avidity score as determined herein in accordance with the invention, it is understood that this takes into account the number of cells that have remained attached after applying the force and which may also take into account one or more markers associated with synapse formation. Hence, as already outlined above, as compared with a cellular avidity score which only takes into account detachment of cells and applying a force, the cellular avidity score in accordance with the invention may provide for a more accurate cellular avidity score which is more reflective of the function of effector cells, i.e. forming synapses with target cells. Hence, the cellular avidity score as determined herein in accordance with the invention may also be referred to as a functional cellular avidity score or a synaptic cellular avidity score. Alternatively, as the result of forming a synapse between an effector cell and a target cell is cell killing, the cellular avidity assay in accordance with the invention may also be an alternative to a potency assay. By measuring markers associated with synapse formation, the most potent, i.e. most strong, cell-cell interaction is determined. Hence, the cellular avidity as determined herein may also be referred to as a cellular avidity potency, and the cellular avidity score referred to as cellular avidity potency score.

In any case, a cellular avidity score can be provided in the methods in accordance with the invention as described herein which allows for a relative measure of cellular avidity which is highly useful, as it allows for a comparison, e.g. in quality control relative to a reference or when comparing different effector cells or the like. It is understood that it is not required to determine the total number(s) of each and every fraction, or representative portion thereof, but that it can suffice to determine the number of cells in a representative portion of a fraction to determine the numbers therein and calculate a score. It is understood that the number of cells in a fraction (or portion thereof) can be determined in situ as well as in collected fractions.

In a further embodiment, a method is provided wherein the effector cells that remain bound with the target cells after exerting a force, e.g. at the end of step d), are subsequently collected. Effector cells that remained bound with target cells and attached with the surface can be mechanically collected, e.g. by scraping of the cells from the surface, or by resuspending the cells. This may be done with a suitable suspension buffer or the like and/or by using high shear force flows. The cells thus obtained can comprise doublets and singlets. Hence, in a further embodiment, the effector cells are collected bound to the target cells. Effector cells bound with target cells (as depicted in FIG. 2, (ii) and (iii) can easily be separated from singlets present, e.g. via cell sorting. Hence, in another embodiment, the effector cells bound to the target cells are separated from the effector cells and/or target cells that did not remain bound after the step of exerting a force (step d).

In a further embodiment, the effector cells that remain bound with the target cells after exerting a force are subjected to a separation step, i.e. the effector cells are detached from the target cells. Accordingly, in this embodiment, effector cells bound with target cells after step d) of applying the force are separated from each other, preferably with trypsin. As shown in the examples, it was found that highly surprisingly, effector cells and target cells that formed a bond, of which a substantial portion is understood to comprise synapses, could be separated from each other with trypsin or the like. Hence, in accordance with this embodiment, the target cells and effector cells that are bound to each other are enzymatically or chemically separated, preferably with trypsin or the like. As shown in FIG. 3, such separation can occur either in situ, i.e. directly on the cells that remained bound to the attached cells after exerting a force (FIG. 3B) or on the cells collected therefrom (FIG. 3A). Either way, single cells can be obtained of target cells and effector cells derived from target cells bound to effector cells that formed e.g. a synapse.

In any case, in the cells obtained, which can be either in the form of target cells bound to effector cells or separated cells, e.g. with trypsin, the marker associated with synapse formation can be determined, which may be in situ or in collected cells. Thus, in a further embodiment, the marker associated with synapse formation is determined in either the target cell or the effector cell. In another embodiment, in the method in accordance with the invention one or more markers associated with synapse formation are determined and the one or more markers are determined in the effector cells and/or in the target cells. When cells are labelled, either in a singlet state or doublet state, cells may highly advantageously be sorted and collected and subsequently analysed.

In one embodiment, a method in accordance with the invention is provided, wherein the marker associated with synapse formation is selected from the group consisting of calcium signalling signatures; spatial clustering of synapse localized molecules such as LFA-1, CD28, CD3, Agrin; changes to internal cell structure and/or cytoskeleton such as F-Actin, Talin, microtubules, centrosome, lytic granules, nucleus position, mitochondrial relocation; changes in effector cell motility; changes in external cell morphology and/or cell shape; and apoptosis of target cells. Synapse formation includes the initiation of a synapse up to and including the establishment of a synapse and subsequent signalling cascade in which, as described above but not necessarily limited thereto, markers associated with synapse formation are associated.

In a further embodiment, the marker for synapse formation is calcium signalling, which can be detected with fluorescent calcium indicators, such as Fura2 AM (available from Invitrogen, item nr. F1221), which marker is suitable for detection in effector cells and in other live cells. Other suitable dyes to detect calcium signalling can be selected from the group of Fura Red AM, Indo-1, pentapotassium, Fluo-3, fluo-4, Calcium Green-1, Rhod-2 and X-Rhod-1, Oregon Green 488 BAPTA. Hence, in one embodiment, the marker for synapse formation is calcium signalling which is detected with an indicator selected from the group consisting of Fura2 AM, Fura Red AM, Indo-1, pentapotassium, Fluo-3, fluo-4, Calcium Green-1, Rhod-2 and X-Rhod-1, and Oregon Green 488 BAPTA. With these markers calcium signalling can be detected which is a hallmark of synapse formation.

Furthermore, membrane potential dyes may also be used as calcium signalling indicators, such as the Invitrogen FluoVolt™ Membrane Potential Kit (Catalog number: F10488). Depolarization of the synapse forming cells using a slow-response potential-sensitive probe such as Invitrogen DiSBAC2(3) (Bis-(1,3-Diethylthiobarbituric Acid) Trimethine Oxonol, Catalog number: B413) may also be used. Hence, in another embodiment the marker for synapse formation is detected utilizing membrane potential dyes.

In another embodiment, the marker for synapse formation is cytoskeleton rearrangement, which can be detected in effector cells with live or fixed cells, using cell staining, e.g. F-actin can be detected with Phalloidin conjugates or CellMask™ (Invitrogen, item nr A57243). Other usable stains can include SiR-Actin, CellLight™ Talin-GFP, BacMam 2.0, or Tubulin Tracker Deep Red. Hence, in one embodiment, the marker for synapse formation is cytoskeleton rearrangement, which is for example detected with a stain selected from the group consisting of Phalloidin conjugates, CellMask™, SiR-Actin, Cell Light™ Talin-GFP, BacMam 2.0, or Tubulin Tracker Deep Red. With these markers, cytoskeleton rearrangement can be detected.

In another embodiment, the marker for synapse formation involves monitoring effector cell motility. Detection of effector cell motility can be performed by video/timelapse monitoring of effector cells that remain in contact with the target cells after the force has been exerted. Detection of synapse formation includes detecting effector cell immobility, i.e. upon synapse formation an effector cell will stop moving and remains into contact with the target cell with which it forms a synapse. Cell motility can be detected by membrane staining of effector cells and imaging, or using, for example, brightfield, darkfield or phase contrast microscopy.

It is understood that for some of the methods for detecting a marker for synapse formation which use e.g. microscopy and monitoring of motility, or short-lived signals, may be combined with having cells provided with e.g. photoactivatable label, to, upon detection of the marker, activate such a label in the cell, such that in subsequent steps, e.g. sorting, FACS analysis or the like, cells for which the marker associated with synapse formation was detected can easily be tracked.

As described above, in accordance with the invention, markers associated with synapse formation can be determined, e.g. via utilizing labels or the like. However, it may be highly advantageous to sequence obtained cells and identify synapse formation by sequencing. It is understood that sequencing comprises nucleotide sequencing, e.g. sequencing of DNA and/or RNA as expressed present in cells. Means and methods are widely known in the art to sequence DNA and/or RNA as expressed in the cell. Hence in another embodiment, in the methods in accordance with the invention the markers are determined with sequencing. This is in particular highly useful for such markers which are up- or downregulated in target cells and/or effector cells in forming a synapse or having a synapse. Suitable markers for T cell activation and synapse formation are transcripts linked to interferon expression, proliferation, and cytokine expression and include: Interferon pathway upregulation: CD4, IFIT3, IFIT2, STAT1, MX1, IRF7, ISG15, IFITM3, OAS2, JAK2, SOCS1, TRIM21; proliferation: LIF, IL2, CENPV, NME1, FABP5, ORC6, GOS2, GCK; cytokine expression: CCL3, IFNG, CCL4, XCL1, XCL2, CSF2, IL10, HOPX, TIM3, LAG3, PRF1, TNFRSF9, NKG7, IL26. Sequencing may also be used to identify non-synapse forming cells by detecting molecular signatures linked to resting cell states, such as: FOXP3, CTLA4, MTNFRSF4, IRF4, BATF, TNFRSF18, TOX2, PRDMI, LEF1, ATM, SELL, KLF2, ITGA6, IL7R, CD52, S100A4, TGFB3, AQP3, NLRP3, KLF2, ITGB7.

It may be also advantageous to determine the molecular state of the target cells as this can give an indication of the cell killing potency of the effector cells. In the case of paired analysis where effector-target cell doublets are recovered, this enables direct linking of effector cell phenotype with their killing capabilities. Next to staining methods for apoptosis commonly used in the field, sequencing or qPCR can be used to detect transcripts linked to apoptosis or cell survival in the target cell, markers include: Bcl-2 family (BCL-XL) caspases 3, caspases 7, cleaved PARP, bax, bad, bak, bid, puma noxa, bcl-2, bcl-xl, mcl-1, p53, and cytochrome c, Smac/Diablo, survivn, Mcl-1, RNA Y1.

As it is understood that changes in gene expression may take some time and are not necessarily immediately detectable, one may allow after the step of exerting the force, the cells to remain bound to the target cells for some time and have these remain attached to the surface as well. Alternatively, one may also separate e.g. doublets that remained after exerting the force and subsequently e.g. sort doublets in single wells, and allow these to remain for some time. A suitable time to allow for expression of markers associated with synapse formation may be from 1 hour to 24 hours.

This is highly advantageous as sequencing methods allow for single cell sequencing, which also allows for determining the sequences of receptors expressed by the cell as well. Hence, in a further embodiment, different receptors are determined and identified via sequencing. In yet another embodiment, sequencing comprises single cell sequencing. In still another embodiment, sequencing comprises sequencing the expressed genome. Sequencing the expressed genome is highly useful as it allows to determine up- and downregulated gene expression. Nevertheless, sequencing genomic DNA may be useful as it may also provide useful information e.g. epigenetic markers associated with in vivo potency and durable response.

In a further embodiment, the applied force in the means and methods of the invention is a force ramp, preferably a linear force ramp. It is understood that the force that is to be applied can be a constant force applied for a defined period. The forces applied may be in various forms as a function of time. Preferably however, the applied force is an increasing force, that is, after the incubation step, an increasing force is applied for a defined period until a defined end force is reached. Increasing the force is preferably done as a linear force ramp, but other ways to increase the force over time may also be used e.g. exponential loading where the force is doubled over a certain small time period and keeps doubling until an end force is reached. For example, as shown in the example section, in about 150 seconds, a defined end force is reached of 1000 pN. An increasing force may be referred to as a force ramp, which may preferably be a linear force ramp, but other ways of increasing the force may be contemplated. As said, detached cells can be collected in a fraction. It may also be opted to collect cells in different fractions, e.g. each fraction representing a defined period in which the force (ramp) was applied and/or a defined force regime. By collecting different detached fractions, one may e.g. construct a cellular avidity curve, i.e. plotting determined fractions at the y-axis, while plotting increasing applied forces at the x-axis.

Any suitable force application method may be contemplated in accordance with the invention. Increasing the force can be well controlled with acceleration-based methods of applying force such as centrifugation, with shear flow and with acoustic force, which are all suitable means to be used in the methods in accordance with the invention but any other means of controllably causing a force on the cells attached to the target cells, thereby forcing them away from the target cells or the functionalized wall surface, may be contemplated. Accordingly, in a further embodiment, the force that is applied is selected from an acoustic force, a shear flow force or an acceleration force such as a centrifugal force.

It may be advantageous to apply shear flow and/or centrifugal flow in scenarios wherein a large number of cells are to be tested. Acoustic force, though very well suitable in the means and methods of the invention, allows for the processing of about 100-10,000 cells, e.g. utilizing a device like the z-Movi® as available from LUMICKS. This is because the surface area at which the acoustic force may be well controlled can be limited. However, using centrifugal force and/or shear flow, the forces exerted on the cells that attach to the target cell layer can be more easily controlled over much larger areas allowing for the analysing and/or sorting of millions to billions of cells.

With regard to the cellular avidity, it is understood that this is indicative of the strength of binding of cells of interest, i.e. cells carrying a receptor (or control cells thereof) to the target cells. It is understood that where we refer to specific forces applied to cells this may refer to average forces, e.g. such forces may not be fully homogeneous, for example over the contact surface as may be the case with acoustic forces and shear-flow forces (see e.g. Nguyen, A., Brandt, M., Muenker, T. M., & Betz, T. (2021). Lab on a Chip, 21(10), 1929-1947) for a description of force inhomogeneities in acoustic force application).

Also, for shear-flow forces the forces may not be fully homogeneous, for example since the flow speed near the side walls of a flow channel (e.g. with a rectangular cross section) may be lower than in the centre of the flow cell (due to the no-slip boundary condition). By choosing a cross section with a high aspect ratio (low and wide), these flow effects may be minimized such that only a few percent of the cells experience a substantially smaller force than the cells in the centre of the flow cell. Other methods to mitigate such effects and to specifically select cells that have experienced similar forces may include using flow cell geometries with multiple fluid inlets and/or outlets such that the properties of laminar flow can be used to ensure cells of interest only land in regions of homogeneous force and/or are only selected from regions of homogeneous force. In one example, by using three channel inlets side by side one can use the side channels as sheath flow channels to focus cells of interest inserted into the central channel inlet towards the centre of the interaction region where the acoustic and/or shear force may be substantially homogeneous. The sheath flow fluid may be the same buffer fluid as is used for the sample cells but then free of sample cells. By increasing the flow speed through the sheath flow channels, the cells are more focused and confined to the centre of the channel, while by reducing the sheath flow speed the cells are allowed to spread out more. Similarly, on the collection side, flows in three side-by-side collection channels may be controlled to possibly discard cells flowing close to the channel boundaries and only collecting cells from the centre of the channel. By controlling the relative flow speeds of such side channels and the centre channel asymmetrically the location of the effective interaction region of the sorting device can be further controlled and cells that have underwent defined forces can be selected and/or detected.

Further means to enable collection of cells from a specific interaction region (and therefore collection of cells that experienced a defined force) include means and methods wherein cells of interest may be provided with a photoactivatable label which may be subsequently activated by illumination with light of a suitable wavelength only in a well-defined interaction region of the device (e.g. near the centre of a flow channel or in a centre region under an (acoustic) force transducer) to photoactivate and/or switch the dye. Subsequently, the cells can be sorted for example using fluorescence activated cell sorting (FACS) and only those cells which are activated are further used according to the methods described herein thereby obtaining the cells on which defined forces have been exerted.

For centrifugal forces, it is easier to ensure that the force applied is homogeneous across the whole interaction region, since such a force does not depend strongly on a location on a surface with respect to a force transducer and/or the wall of a flow channel or sample holder.

Accordingly, in connection to the subject matter disclosed herein, means and methods exist which allow one to exert forces on cells attached to a surface and collect the cells, detached and/or attached cells, on which defined forces have been exerted. It is understood that with regard to the force exerted, the exact forces experienced by cells may also depend on cell size and or other cell properties such as density and compressibility. The force may be a nominal force and not the true force experienced by the cells. E.g. it may be hard to precisely predict the average cell size, density, compressibility, etc. of the cells and the force may have been calculated based on theory alone or may have been calibrated using test particles with specific (preferably known) properties (see e.g. Kamsma, D., Creyghton, R., Sitters, G., Wuite, G. J. L., & Peterman, E. J. G. (2016). Tuning the Music: Acoustic Force Spectroscopy (AFS) 2.0. Methods, 105, 26-33). The force may be such a calculated or calibrated force expressed with units of N (e.g. pN), but it may also be expressed without calibration as the input power (Vpp) applied to a piezo element (see Sitters, G., Kamsma, D., Thalhammer, G., Ritsch-Marte, M., Peterman, E. J. G., & Wuite, G. J. L. (2014), Acoustic force spectroscopy. Nature Methods, 12(1), 47-50), as angular velocity squared (ω²) in the case of centrifugal force application or as flow speed v and or as shear stress (Pa) in applications using shear forces. As long as the forces exerted by the devices, e.g. shear force, acoustic force, or centrifugal forces, but not limited thereto, can be varied and controlled and reproduced in such devices, such devices are suitable for the means and methods in accordance with the invention.

In another embodiment, the target cells are attached to a glass or a plastic surface, preferably a glass surface in a chip or the like (such as described i.a. in Fernández de Larrea, C. et al. (2020). Blood Cancer Discovery, 1(2), 146-154) with target cells attached to its surface. Such a chip is also described in U.S. Pat. No. 10,941,437B2 and WO2018083193. The target cells that are attached to the surface, preferably are attached as a monolayer. The monolayer preferably is at high confluency. The subsequent cells that are to interact with the target cells are preferably provided in a relatively low cell density as compared with the target cells, such that substantially all cells can interact with a target cell (there are more target cells available for the total number of cells comprised in the effector cells). Such provides for advantageous controllable conditions when applying the force on the cells, e.g. on the effector cells.

As said, the target cells may be cancer cells or cells presenting an antigen. With regard to effector cells, immune cells are contemplated in accordance with the invention. In a preferred embodiment immune effector cells are provided as effector cells with a receptor, and target cells expressing an antigen which the cell of interest is to specifically target. For example, effector cells may be selected from T cells, NK cells, dendritic cells, neutrophils, macrophages, or monocytes. Such cells may be genetically modified, e.g. provided with e.g. a CAR. Preferably, effector cells may be CD3+ T cells.

As said, target cells are typically cancer cells, or other cells expressing an antigen, e.g. viral antigens or the like. Hence, in yet another further embodiment, the receptor is a CAR or a TCR.

Further embodiments include methods being performed in the presence of agent(s). For example, one or more agents capable of modulating the cellular avidity between an effector cell with a receptor and a target cell may be included in the steps of contacting effector cells with target cells and subsequently applying a force away from the target cells. Modulating is understood to either enhance or reduce cellular avidity. This way, for example, in case an agent is present that is known to modulate a desired interaction between a cell with a receptor and a target cell, receptor/target interactions may be selected that are not affected by such agents, or, conversely, are aided by such agents. Hence, in another embodiment, an agent capable of modulating the interaction between the effector cells with a receptor and the target cell is included at least in the contacting step and when applying a force. For example, an agent could be used that reduces aspecific binding between a cell with a receptor and target cells.

In another embodiment, a cell engager may be provided. Cell engagers include antibodies, or the like, which are capable of binding to a target cell and an effector cell. Such antibodies may include single chain antibodies comprising two binding domains such as scFv domain, and include BiTEs (i.e. bispecific T cell engagers) or the like. A conventional antibody design includes heavy and light chains, with one half of the antibody (one heavy chain and one light chain) engaging with a target cell, and the other half of the antibody (another heavy chain and another light chain) engaging with an effector cell, wherein preferably, the Fc domain is made inert. In any case, suitable cell engagers are widely known in the art and the current invention allows to study and/or determine cellular avidity, e.g. synapse formation, induced by a cell engager between a target cell and an effector cell. Hence, accordingly, in the means and methods in accordance with the invention, a cell engager may be provided in addition, and e.g. the cellular avidity score is determined, induced by said cell engager between an effector cell and a target cell, said cell engager having a binding region capable of binding the effector cell and a binding region capable of binding the target cell. It is understood that the contacting step in the methods of the invention can thus be performed in the presence of the cell engager to allow the cells to interact, i.e. effector cells and target cells, and form a bond, e.g. a synapse, via the cell engager. Accordingly, in one embodiment, in methods in accordance with the invention, a cell engager is provided capable of binding the effector cell and the target cell and inducing synapse formation, and the cell engager is included in the contacting/interacting step. Furthermore, such methods are highly useful for screening cell engagers, e.g. by identifying cell engagers that are particular capable of inducing a synapse.

It was observed that when cells that remained bound and attached after a cellular avidity measurement were resuspended utilizing i.a. repeated pipetting and thus exerting a significant force, such as described e.g. in the example section, cells attached to the surface were detached and moreover, a portion of the cells that were bound to the attached cells became unbound. Hence, this implied that with such a process step a differential force can be applied on bound cells that can exceed the maximum force that is applied away from cells attached to a surface. This way, further cell-cell bonds that may be aspecific cell-cell bonds may thus be broken therewith providing substantially effector cells bound with target cells (and optionally a cell engager) that formed a synapse can be retained. Hence, in a further embodiment, after the force has been applied away from the attached cells, subsequently, the cells are resuspended and a differential force is applied such that substantially cells that are bound to each other via a synapse remain bound to each other and substantially cells that have formed aspecific bonds are unbound, i.e. have their cell-cell bonds broken. It is also understood that instead of applying the force away, the differential force may be applied instead, and that by applying a differential force, it may no longer be required to have cells attached to a surface, i.e. immobilized. Furthermore, it is also understood that after the differential force has been applied, cells are highly preferably separated and/or sorted in order to avoid further interaction between cells to avoid establishing additional cell-cell bonds. Hence, highly preferably, cells are collected and sorted and/or analysed after the differential force has been applied.

As is clear from the above, the type of force that is to be applied in accordance with the invention is a force capable of breaking cell-cell bonds, i.e. the force exerted causes cells bound to each other to move away from each other to such an extent that a cell-cell bond may break or rupture. A differential force means that the force on one cell differs from the force on the other cell with regard to direction of the force and/or the magnitude of the force, resulting in a net force allowing to break cell-cell bonds if the differential force exceeds the binding force.

For example, when a target cell bound to an effector cell, a doublet, is forced through a nozzle, the closer to the throat of the nozzle the faster the flow. This means that the first cell to enter the nozzle is subjected to a stronger acceleration than the cell lagging behind and the cells experience a differential force resulting in a net force which can result in cell-cell bond rupture, provided the force is large enough (see e.g. FIG. 5, in particular 5c). A differential force that can be applied includes a shear force, e.g. such as can be applied utilizing repeated pipetting (repeated upwards and downwards flow of the sample) or flow through a nozzle.

Other means and methods are known in the art with which shear forces can be applied to cells, e.g. flowing a cell suspension at a constant speed and bombarding these cells to a flat surface at a defined angle. Furthermore, forcing a cell suspension through a needle with a defined internal diameter and a defined force may provide for a well controllable shear force as well. The cell suspension may be subjected to several rounds of such process steps to ensure substantially all cell-cell bonds experience the maximum force that may be achieved with the process step. Such a process step allows for automation, enabling control and repeatability of the process, therewith controlling shear forces exerted. Suitable devices for breaking apart cell-cell bonds which are not synapses are known in the art (e.g. Zahniser et al., J. Histochem. Cytochem. 1979, 27(1), 635-641). Also, by properly tuning the forces in a flow-cytometer normally used to measure cell deformations, such as e.g. described in Otto, et al. Nat. Methods 12, 199-202(2015) suitable forces can be applied. Tuning can be achieved e.g. by changing the nozzle size or geometry and/or the flow speeds used. Other suitable devices known in the art may include for a vortex mixer, with which shear forces may be suitably applied as well. Accordingly, in one embodiment, the force applied involves a shear force.

In another embodiment, the force applied is an ultrasonic force. It is understood, as outline above, that such ultrasonic forces are not forces such as applied e.g. in a device as available from LUMICKS, wherein the force is away from attached cells (e.g. such as in the LUMICKS z-Movi® Cell Avidity Analyzer, e.g. as used by Larson et al., Nature 604, 7906:1-8, Apr. 13, 2022). It is also understood that the ultrasonic force is selected such that cells are not lysed. Hence, appropriate ultrasonic forces can be applied to cells such that cell-cell bonds can be ruptured, which more preferably includes breaking aspecific cell-cell bonds and less preferably breaks specific cell-cell bonds in which an immune synapse is formed. Examples of using ultrasonic forces to break (aspecific) cell-cell bonds, are known in the art (e.g. as described in Buddy et al., Biomaterials Science: An Introduction to Materials in Medicine, 3^{rd} edition, 2013, Chapter 11.2.8, page 576; and Moore et al., Experimental Cell Research, Volume 65, Issue 1, 1971, Pages 228-232).

In any case, suitable applied forces which are known in the art include e.g. a force in the range of 50 pN-10 nN, which said force is a net force exerted on one cell relative to the other cell, of two cells bound to each other. Which means the force is exerted on the cell-cell bond. In another embodiment, the force exerted on one of the two cells relative to the other cell is at least 50 pN, or at least 100 pN, or at least least 200 pN. In another embodiment, the force exerted is at most 10 nN, at most 5 nN, at most 3 nM, at most 2 nM, or at most 1 nN. In yet another embodiment, the force is selected from the range of 1 pN-10 nN, from 100 pN-10 nN, from 500 pN-10 nN, from 1 nN-10 nN. In still a further embodiment, the force is selected from the range of 500 pN-5 nN, from 500 pN-4 pN, from 500 pN-3 pN. For example, a suitable amount of force that can be exerted between cells (e.g. such as in the z-Movi® device) can be selected to be in the range of 200 pN-3000 pN. Of course, these force ranges are known to be useful with cells attached to a surface, and the maximum force that may be selected may exceed 3000 pN as it is not required to have the cells attached to a surface in accordance with the invention.

Accordingly, it is understood that in these embodiments, the differential force to be applied does not require either of the target cells or effector cells to be attached, and the differential force is a force selected from the range of 50 pN-10 nN. In another embodiment, in methods in accordance with the invention wherein the force that is applied is a differential force, neither the target cells nor the effector cells require to be attached to a surface, and the differential force is applied in the range of 50 pN-10 nN, thereby providing cells substantially comprised of target cells, effector cells, and effector cells bound with target cells via a synapse.

Without being bound by theory, as is understood the range of force that may break an aspecific cell-cell bond versus a specific cell-cell bond that forms a synapse differs. This difference can be to such an extent that the ranges of the required forces do not overlap. It is understood that some overlap may occur. Hence the force that is selected, as outline above, may allow for aspecific cell-cell bonds remaining and some specific cell-cell bonds that formed a synapse to break. In case there is substantially no overlap, a differential force can be selected, as outline above, which allows substantially for aspecific cell-cell bonds to break, while substantially retaining specific cell-cell bonds that formed a synapse. In case there is no overlap, and ranges are sufficiently far apart, a differential force may be selected, as outline above, which allows for aspecific cell-cell bonds to break while retaining specific cell-cell bonds that formed a synapse. As also outlined herein, the portion of cell-cell bonds that remains after exerting a force and has a synapse, can be determined by determining the presence or absence of a marker associated with synapse formation.

In one embodiment, after the step of applying a force in the methods of the invention, the cells are resuspended and a subsequent differential force is applied such that formed aspecific cell-cell bonds are broken. In another embodiment, in methods of the invention wherein either the target cells or effector cells (carrying a receptor) are attached to a surface, this attachment is optional, and, in the step of applying the force instead a differential force is applied. In yet another embodiment, in methods of the invention wherein either the target cells or effector cells are attached to a surface, this attachment is optional, and, in the step of applying the force instead a differential force is applied, wherein the differential force is such that cells that are bound via a synapse remain bound to each other and formed aspecific cell-cell bonds are broken.

The present application also describes a method for detecting the presence or absence of a marker associated with synapse formation, comprising the steps of:
a) providing effector cells carrying a receptor;
b) providing target cells;
wherein the effector cells carrying a receptor are capable of binding target cells, and wherein the effector cells or the target cells are attached to a surface;
c) contacting the effector cells with the target cells to allow the cells to interact with each other;
d) applying a force away from the cells attached to the surface, such that at least part of the cells bound to the cells attached to the surface move away therefrom;
e) detecting effector cells that have remained bound with the target cells and attached to the surface after applying the force and the presence or absence of a marker associated with synapse formation.

The present application also describes a method for identifying a receptor capable of binding target cells and inducing synapse formation, from a heterogeneous population of effector cells comprising a plurality of effector cells carrying a variety of receptors, comprising the steps of:
a) providing the heterogeneous population of effector cells;
b) providing target cells;
wherein target cells or heterogeneous population of effector cells are attached to a surface,
c) contacting the plurality of effector cells carrying the receptor with the target cells to allow the cells to interact with each other;
d) applying a force away from the cells attached to the surface, such that at least part of the cells bound to the cells attached to the surface move away therefrom;
e) detecting effector cells carrying a receptor that remain attached to target cells having a marker associated with synapse formation and determine from effector cells associated with said marker the receptor thereby identifying a receptor capable of binding target cells and inducing synapse formation.

The present application also describes a method for identifying a receptor capable of binding target cells and inducing synapse formation, from a plurality of effector cells carrying a variety of receptors, comprising the steps of:
a) providing a plurality of effector cells carrying a variety of receptors;
b) providing target cells attached to a surface;
c) contacting the plurality of effector cells carrying the receptor with the target cells to allow the cells to interact with each other;
d) applying a force on the plurality of effector cells carrying a variety of receptors away from the target cells, such that at least part of the plurality of effector cells carrying a variety of receptors detach and/or move away from the target cells;
e) detecting effector cells carrying a receptor that remain attached to the target cells, having a marker associated with synapse formation, and determine from effector cells associated with said marker the receptor thereby identifying a receptor capable of binding target cells and inducing synapse formation.

The present application also describes a method in accordance with any one of the previously described methods, wherein a cellular avidity score is determined for identified receptors having a marker associated with synapse formation.

The present application also describes a method in accordance with any one of the previously described methods, wherein the effector cells that remain bound with the target cells after step d) of applying the force are subsequently collected.

The present application also describes a method in accordance with any one of the previously described methods, wherein the effector cells bound to the target cells are separated from the effector cells and/or target cells that did not remain bound after step d) of applying the force.

The present application also describes a method in accordance with any one of the previously described methods, wherein effector cells bound with target cells after step d) of applying the force are separated from each other, preferably with trypsin.

The present application also describes a method in accordance with any one of the previously described methods, wherein one or more markers associated with synapse formation are determined and wherein the one or more markers are determined in the effector cells and/or in the target cells.

The present application also describes a method in accordance with the previously described method, wherein the marker associated with synapse formation is selected from the group consisting of calcium signalling markers; spatial clustering of synapse localized molecules such as LFA-1, CD28, CD3, and Agrin; changes to internal cell structure and/or cytoskeleton such as F-Actin, Talin, microtubules, centrosome, lytic granules, nucleus position, and mitochondrial relocation; changes in effector cell motility; changes in external cell morphology and/or cell shape; and apoptosis of target cells.

The present application also describes a method in accordance with any one of the previously described methods, wherein the markers are determined by sequencing or staining.

The present application also describes a method in accordance with any one of the previously described methods, wherein the applied force is a force ramp, preferably a linear force ramp, and wherein the applied force is an acoustic force, a shear flow force or an acceleration force.

The present application also describes a method in accordance with any one of the previously described methods, wherein the target cells are cancer cells or cells presenting a cancer antigen, and wherein the effector cells carrying a receptor are selected from T lymphocytes, NK cells, monocytes, neutrophils, macrophages and dendritic cells, and wherein optionally a cell engager is provided at least in the contacting step c).

The present application also describes a method in accordance with any one of the previously described methods, wherein after the step of applying a force in step d) the cells are resuspended and a differential force is applied such that formed aspecific cell-cell bonds are broken.

The present application also describes a method in accordance with any one of the previously described methods, wherein said attachment of cells is optional, and, in the step of applying the force away, instead a differential force is applied.

The present application also describes a method in accordance with the previously described method, wherein the differential force is such that cells that are bound to each other via a synapse remain bound to each other and formed aspecific cell-cell bonds are broken.

### EXAMPLES

### Example 1

### Synapse Detection Using Calcium Imaging

### Calcium Imaging

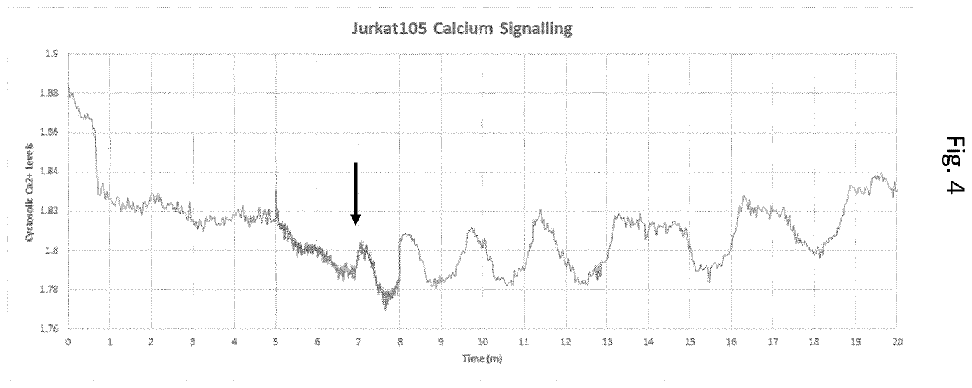

FIG. 4 shows a trace of the cytosolic Ca²⁺ levels of a NALM6 target cell during an avidity measurement. A LUMICKS z-Movi® chip was prepared by coating glass with Poly-L-Lysine. Nalm6 target cells were seeded in monolayers inside z-Movi® chip channels. Effector cells, Jurkat 105 cells (FMC63 CAR receptor specific for CD19 expressed on the surface of NALM 6 cells) were loaded with FuraRed (thermo) dye suitable for calcium imaging by incubating with 2.5 μM FuraRed in complete RPMI for 45 minutes at 37° C.

The chip was placed on a Nikon Ti2 widefield fluorescence microscope and fluorescence images were recorded in 630/70 emission using 430 and 500 excitation at 25% power. At time 0 the effector cells were loaded into the chip and allowed to incubate onto the target cells. The Ca²⁺ level in the cells was monitored by ratio metric intensity (R430/500) using the Nikon Elements software as described e.g. by Gregg et al., J Biol Chem, 2019 Mar. 22; 294(12): 4656-4666.

After 5 minutes of incubation, a 150 second linear force ramp (0-1000 pN relative force) was applied to the effector cells using the acoustic actuator. Effector cells that were not strongly bound to the target cells were lifted off and pushed towards the acoustic nodes of the z-Movi® chip. Of the cells that remained bound, some cells showed clear calcium oscillations as visible in FIG. 4 from minute 7 (marked also with a black arrow) to minute 20. These calcium oscillations indicate internal signalling activity associated with antigen specific binding and T cell activation. In the fluorescence microscopy video (not shown) of the same target cell it was observed that the Jurkat cell clearly wraps around or engulfs the NALM6 cell. Such engulfment is characteristic of synapse formation confirming that indeed the calcium oscillations can be used as a marker for synapse formation in combination with cell avidity experiments.

### Membrane Potential Dyes

Since T cell killing through perforins and apoptosis signalling results in membrane permeability and depolarization of the target cells, other possible markers for synapse formation are membrane potential dyes. These dyes can be used to visualize the change in membrane potential in target cells with an immune synapse and therefore can be used to distinguish between synapse forming cells and cells that remain stuck through background binding. An example of a membrane potential dye that may be suitable is the Invitrogen FluoVolt™ Membrane Potential Kit (Catalog number: F10488). It may be preferred to detect the depolarization of the synapse forming cells by using a slow-response potential-sensitive probe such as Invitrogen DiSBAC2(3) (Bis-(1,3-Diethylthiobarbituric Acid) Trimethine Oxonol), Catalog number: B413. In this case, increased fluorescence would indicate the target cell has depolarized and the bound effector cell formed a synapse effectively initiating the target cell killing cascade.

### Example 2

### CD3ζ Phosphorylation

A z-Movi® cell avidity experiment is performed according to the standard protocol including 10-15 minutes of incubation of T cells on a target cell monolayer and a 150 second linear force ramp (0-1000 pN relative force) using the acoustic actuator. Immediately following the force ramp, unbound cells are flushed out and bound cells are fixed inside the z-Movi® chip using BD Phosflow Fix Buffer I (Cat. No. 557870) at 37° C. for 10 minutes and permeabilized in BD Phosflow Perm Buffer III (Cat. No. 558050) on ice for at least 30 minutes and stained with Alexa Fluor® 488 Mouse anti-CD247 (pY142, Clone K25-407.69, BD). The z-Movi® chip is subsequently imaged and activated cells are identified based on their Alexa Fluor® 488 brightness. Avidity data can now be combined with the CD3ζ phosphorylation data to improve avidity data and gain more insight e.g. into T cell potency.

### Example 3

In another experiment, following 10-15 minutes of incubation of T cells on a target cell monolayer, a force ramp is applied in a centrifugal avidity apparatus, and all cells are flushed out immediately with force. This provides for a mixture of monolayer cells, effector cells and effector-targeT cell doublets. The obtained cell suspension is fixed using BD Phosflow Fix Buffer I (Cat. No. 557870) at 37° C. for 10 minutes and permeabilized in BD Phosflow Perm Buffer III (Cat. No. 558050) on ice for at least 30 minutes and stained with Alexa Fluor® 488 Mouse anti-CD247 (pY142, Clone K25-407.69, BD). The stained cell suspension is subsequently analyzed and single-cell index sorted into 384 well plates. The FACS data will allow to determine the fractions of activated (CD32 phosphorylated), and not activated effector cells. These can occur either as singlets or as effector-targeT cell doublets. Single cell RNA-sequencing of the sorted effector cells and doublets will provide information that allows linking the phenotypes of the effector cells to their ability to be activated and form a synapse. This method is not limited to using individually sorted cells as an anti-CD247 antibody linked to a DNA barcode can be used allowing cells to be processed for single-cell sequencing libraries on a 10× genomics platform or other similar platforms.

### Example 4

### F-Actin Live Imaging

A z-Movi® cell avidity experiment is performed according to standard protocols with the following changes: effector cells are preincubated with both Cell trace far red cell tracking dye as well as CellMask™ green actin stain for 30 minutes at 37° C. and 5% CO₂. Optionally a nuclear stain can also be added, e.g. Hoechst 34580. Effector cells are then washed twice and introduced into the z-Movi® chip in a buffer containing CellMask™ dye. After the avidity experiment is performed, unbound effector cells are flushed out and media is replaced with imaging buffer containing no dye. Both brightfield, and fluorescence images in red channel and green channel are acquired, to image the monolayer, the CellTrace stained effector cells, and the F-actin structure respectively. F-actin structure and localization to the synapse interface can now be used to improve the avidity measurement by excluding bound cells that did not show cytoskeletal synaptic structures. The size and quality of the F-actin structure in effector cells may also be used to obtain additional information on the potency of the effector cells.

### Example 5

Transcriptomic Sequencing (e.g. Markers NME1, IL2RA, IFIT3)

An avidity experiment is performed using centrifugal based force application on a target cell monolayer incubated with effector cells. After force application (>600G (>200pN), >1 minute), unbound cells are flushed out from the flow cell and the medium is replaced. The flow cell is returned to a cell culture incubator and incubated for 2-24 hours, preferably 4-8 hours at 37° C. During this incubation activated cells will change their RNA expression from their resting state to one indicating activation and synapse formation. After incubation, all cells are recovered from the device using a fluidic pulse, immediately chilled on ice and FACS sorted to remove target cells. Effector cells may be directly sorted as single cells into well plates for sequencing preparation or pooled for processing on a 10× genomics platform or simply used for bulk sequencing. Sequencing data may now be used to determine the fraction or level of activation in the bound cell population as well as to gain understanding in the specific phenotypes that are present in the bound effector cells. Transcriptomic data adds more information to the avidity measurement and may improve potency prediction of the assay.

### Example 6

### Doublet and Singlet Retrieval & Breaking Cell-Cell Bonds and Trypsin Treatment

A monolayer of target Nalm6 (CD19+) cells (obtained from ATCC, product nr. CRL-1567) was brought in contact with IL-2 stimulated primary human effector T cells purified from buffy coat, which were transduced with CAR-FMC63 anti-CD19 (Kramer, AM; (2017) Delineating the impact of binding-domain affinity and kinetic properties on Chimeric Antigen Receptor T cell function. Doctoral thesis, UCL (University College London)). After removal of free and unbound cells by washing and centrifugation, cells remaining bound were resuspended and optionally trypsinized and subjected to FACS analysis.

### Monolayer Seeding

Nalm6 (CD19+) target cells were used, which were seeded as a monolayer to the ceiling of a 400 μm tall channel slide (μ-slides obtained from Ibidi, Cat. No: 80176). First, cells were counted and resuspended in serum-free medium at a concentration of approximately 30×10⁶ cells/mL in order to acquire a confluence close to 100%. Next, 100 μL of the resuspended cells was pipetted into the inlet of the channel slide and introduced in the channel by tilting the slide for a few seconds until it reached the outlet of the channel slide. The channel slide was then placed horizontally, so that the 100 μL filled the entire volume of the channel. The channel slide was flipped immediately, and incubated upside down in a humidity, temperature, and CO₂ controlled incubator for 30 minutes. After incubation, the medium was exchanged by first applying 60 μL of serum-containing medium into the inlet and subsequently withdrawing 60 μL from the outlet. This process was repeated four times. The slide was placed back upside down in the controlled incubator and incubated for another 30 minutes. Next, the medium was exchanged with PBS containing 0.5-1 μM CellTrace Violet (ThermoFisher #C34557) and incubated for 15 minutes in the incubator. Finally, the staining solution was exchanged by first applying 60 μL of the serum-containing medium into the inlet and subsequently withdrawing 60 μL from the outlet. This process was repeated four times.

### Effector Cell Preparation

In preparation of subsequent steps, primary T cells transduced with the anti-CD19 CAR-FMC63 were cultured. Cells were counted and viability was tested according to standard protocol. Next, 3×10⁶ cells/mL were stained with PBS containing 1 μM CellTrace Green CMFDA (Thermo Fisher #C2925) and incubated for 15 minutes in the incubator. Finally, the primary T cells were pelleted and resuspended in complete medium.

### Effector Cell Binding

The channel inlet and outlet were filled with plugs prior to centrifugation in order to keep the liquid from moving out of the channel during centrifugation. The channel slide containing the seeded Nalm6 target cells was placed upright into a custom adaptor for the centrifuge bucket and spun for two minutes at 1000×g. Centrifugation removes fractions of the cells that were not well attached to the glass. Next, the slide was removed from the centrifuge. First, 100 μL of the effector cell suspension was pipetted into the inlet, after which 100 μL was withdrawn from the outlet in order to obtain a homogenous distribution of effector cells throughout the length of the channel. The slide containing the target and effector cells was held upside down (to allow the effector cells and Nalm6 cells on the ceiling to interact) and incubated for five minutes.

After incubation, three locations of the channel were imaged (one in the centre and one close to the inlet/outlet) using a fluorescent microscope. Each location was imaged in the brightfield, Green, and Violet channel. The slide was placed back in the centrifuge using the custom adapter and spun for two minutes at 1000×g. Finally, the channel slide was removed from the centrifuge and imaged at the same locations and using the same channels. It was observed that about 50% of the cells remained bound to the target cell after centrifugation. In general, low background binding is observed with Nalm6 cells when applying the centrifuge procedure, which usually is in the range of 5-15%.

### Cell Collection and FACS Analysis

After effector cell binding and imaging, the medium was exchanged by first applying 60 μL of serum-containing medium into the inlet and subsequently withdrawing 60 μl from the outlet. This process was repeated four times. The aliquots of removed medium from the outlet were discarded. Next, two 5 mL syringes were used to flush the target cells and the effector cells which were still attached to the ceiling of the channel slide out of the channel. In order to do so, the plunger of one empty syringe was removed to act as a reservoir. The complete syringe was filled with 3 mL of complete medium and attached to the inlet. The empty syringe with the removed plunge was attached to the outlet. The syringe containing the complete medium was emptied in one vigorous movement, ensuring that all the contents moved through the channel and into the empty syringe. The flush was then reversed by pulling 3 mL back on the plunger of the complete syringe. The contents of the complete syringe were aliquoted into a clean 15 ml tube. The described flush procedure was repeated once more using the same two syringes and the final contents were pooled in the 15 mL tube. The cells in the 15 mL tube were pelleted by centrifugation at 400×g for five minutes. The medium was aspirated/decanted and the pellet was resuspended in serum-free medium.

The cell suspension was aliquoted in two equal parts into two clean tubes and each of the aliquots were pelleted by centrifugation at 400×g for five minutes. One pelleted aliquot was resuspended in 500 μL of Trypsin-EDTA (Thermo Fisher #25300054) and the other in 500 μL PBS.

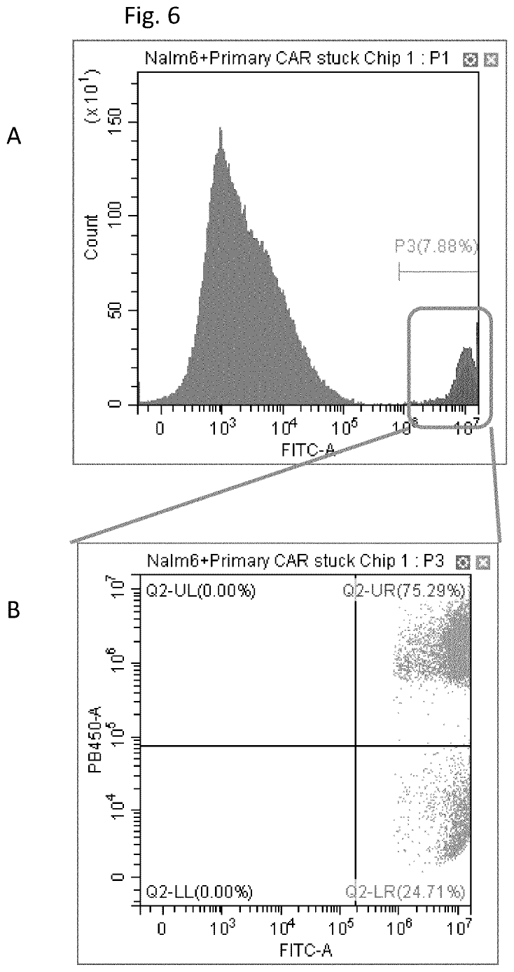

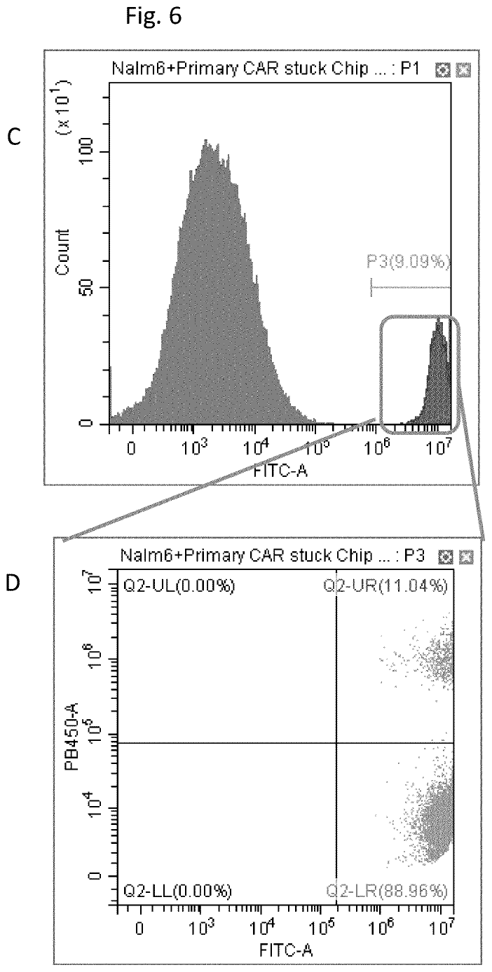

Both resuspended cell pellets were placed in the incubator for 10 minutes. Next, 5 mL of serum-containing medium was added to both tubes to dilute and inactivate the trypsin, if present, and the cells were pelleted by centrifugation at 400×g for five minutes. Both pellets were resuspended in FACS buffer (PBS, 0.5-1% BSA or 5-10% FBS, 0.1% NaN₃ sodium azide) and processed using a Flow Cytometer. Cell trace Violet was detected using a 405 nm laser and a 450/45 BF filter (PB450 channel), while Cell trace Green CMFDA was detected using a 488 nm laser and a 525/40 BF filter (FITC-A channel). See FIG. 6 showing the FACS plots thus obtained, further described below.

Utilizing the Flow Cytometer, cells of either PBS or Trypsin-EDTA treatment were first gated in order to remove debris. Around 100,000 events were gathered. No single cell gate was applied. Detection of the counts (i.e. Cell trace Green CMFDA) revealed a high intensity population in the FITC-A channel that was 7.88% and 9.09% of the total events that passed through the flow cytometer, respectively from the PBS control treated aliquot and the Trypsin treated aliquot (FIGS. 6A and 6C and Table 1). The populations were assumed to comprise single T cells (“singlets”) and T cells bound to a Nalm6 cell (“doublets”). During the second gating, the subpopulation of the first gate was qualified based on the event having a signal in the FITC-A channel only, or in the FITC-A and PB450 channel simultaneously (FIGS. 6B and 6D). Of the PBS control treated aliquot, 75.29% of the counts were gated to the double positive channel (i.e. PB450 and FITC-A), whereas 24.71% were gated to be positive to FITC-A only (see Table 1). Of the Trypsin treated aliquot, only 11.04% were gated to the double positive channel, whereas 88.96% were gated to be positive for FITC-A only (see Table 1). Counts derived from the trypsin treated aliquot presented as viable cells remain viable as judged from FACS analysis, including the observation that cells remained fluorescently labelled.

If we assume that for each aliquot, we started out with 1000 counts, this means that with PBS of the 1000 counts, 79 counts represented T cells, of which 59 T cells were bound to target cells, and 20 were T cells not bound to a target cell. With the Trypsin treated aliquot, 91 counts represented T cells of which 9 were bound, and 82 were not bound to a target cell (see Table 2).

It is remarked that the counts observed in the FITC-A channel are to originate from T cells that remained bound to target cells after centrifugation, and counts being negative for target cells (i.e. PB450-A) were to result from breaking the bond between T cells and target cells after the first centrifugation. From the results, it can be clearly observed that Trypsin-EDTA was able to efficiently cleave the bond between T cells and target cells, which is understood to comprise a highly substantial amount of synapses. Furthermore, because the PBS treated aliquot resulted in a substantial portion of the cells representing T cell singlets, this implies that the procedure of resuspension, and hence the force exerted, which is to exceed the force exerted during centrifugation (because the Nalm6 cells are detached from the channel slide in the procedure) caused further aspecific cell-cell bonds to break between T cells and target cells.

### Example 7

### Transcriptomic Sequencing for Discovery

If a number of effector cells are available, with either natural or engineered differences, e.g. differing by TCR or CAR, it can be beneficial to determine the cellular avidity of a mixed population of these effector cells. A second method e.g. sequencing, PCR or hybridization-based, may then be used to link unique effector cell types to their cellular behaviour.

One application can be the discovery of TCR sequences that result in a high cellular avidity form a population of T cells isolated from blood with a larger diversity of TCR sequences. We describe here a method for a mixed CAR-T cell population containing a library of different CAR-T receptors, e.g. 200 different receptor mutants. An avidity experiment is performed by incubating the library of CAR-T cells on a target cell monolayer for 5-15 minutes and applying a force to separate cells. Immediately following the force application unbound cells are flushed out and collected as the unbound fraction. To detect effector cell activation and synapse formation the media is replaced by one containing antibodies that detect surface markers for activation e.g. CD69, CD137, CD27, CD154/TRAP/CD40L, CD134, and the cells are allowed to incubate in the device for 10 minutes-2 hours. After this period, all remaining cells including the bound effector cells are recovered from the device using a fluidic pulse, and immediately chilled on ice. Effector cells from both the bound and unbound recovered fractions may be directly sorted as single cells into well plates for sequencing preparation or used for processing on a 10× genomics platform. The activation marker antibodies allow FACS sorting to record activation levels for the bound effector cells.

Alternatively, RNA sequencing profiles of the effector cell may also serve as a method to identify activated and synapse forming effector cells. RNA or genomic sequencing for the CAR-T receptor sequence is used to link the individual CAR-T types from the library to avidity and activation status. Efficient detection of TCR or CAR-T receptor sequences is preferably achieved by a targeted sequencing protocol where PCR amplification is performed using primers that enrich for the variable CAR-T or TCR regions. The recovered CAR sequences can be ranked based on their frequencies in the low avidity (unbound) and high avidity (bound) fractions. Performing an experiment this way allows to assess a population of effector cells with mixed or even unknow CAR or TCR sequences and discover how CAR sequence changes are linked to avidity. In some cases, it may be beneficial to mix pure populations of effector cells with different receptors and perform the experiment on the mixed population. In this case prior to mixing individual population may be barcoded e.g. by a DNA barcode oligo linked to an antibody. Such a barcode may facilitate detection of the species in the mixed population by e.g. qPCR, sequencing or other means.

TABLE 1

| | FITC-A (%) | PB450-A (+) (%) | PB450(−) (%) |
|---|---|---|---|
| PBS | 7.88 | 75.29 | 24.71 |
| Trypsin | 9.09 | 11.04 | 88.96 |

TABLE 2

| | FITC-A (%) | PB450-A (+) (%) | PB450(−) (%) |
|---|---|---|---|
| PBS | 79 | 59 | 20 |
| Trypsin | 91 | 9 | 82 |

1. A method for determining the cellular avidity of an effector cell carrying a receptor, capable of binding target cells, comprising the steps of:
a) providing effector cells carrying a receptor;
b) providing target cells;
wherein the effector cells or the target cells are attached to a surface;
c) contacting the effector cells with the target cells to allow the cells to interact with each other;
d) applying a force away from the cells attached to the surface, such that at least part of the cells bound to the cells attached to the surface move away therefrom;
e) detecting effector cells that have remained bound with the target cells and attached to the surface after applying the force, and having a marker associated with synapse formation, and determine the number of effector cells and/or target cells associated with said marker; and,
f) assigning a cellular avidity score based on the determined number of effector cells and/or target cells associated with said marker.

2. A method for determining the cellular avidity of an effector cell carrying a receptor, capable of binding target cells, comprising the steps of:
a) providing effector cells carrying a receptor;
b) providing target cells attached to a surface;
c) contacting the effector cells with the target cells to allow the cells to interact with each other;
d) applying a force on the effector cells away from the target cells, such that at least part of the cells carrying the receptor detach and/or move away from the target cells;
e) detecting effector cells bound to the target cells after applying the force and having a marker associated with synapse formation, and determine the number of effector cells and/or target cells associated with said marker; and
f) assigning a cellular avidity score based on the determined number of effector cells and/or target cells associated with said marker.

3. The method in accordance with claim 1, wherein the number of effector cells bound to target cells after step d) is optionally determined, and wherein the cellular avidity score is determined by calculating the ratio of the number of effector cells carrying the receptor with the marker associated with synapse formation to the number of effector cells bound to the target cells after step d), or, to the number of effector cells that have interacted with the target cells in the contacting step c), or, to the number of effector cells carrying the receptor with the marker associated with synapse formation combined with the number of effector cells carrying the receptor without the marker associated with synapse formation.

4. The method in accordance with claim 1, wherein the effector cells that remain bound with the target cells after step d) of applying the force are subsequently collected.

5. The method in accordance with claim 4, wherein the effector cells are collected bound to the target cells.

6. The method in accordance with claim 5, wherein the effector cells bound to the target cells are separated from the effector cells and/or target cells that did not remain bound after step d) of applying the force.

7. The method in accordance with claim 1, wherein effector cells bound with target cells after step d) of applying the force are separated from each other, preferably with trypsin.

8. The method in accordance with claim 1, wherein one or more markers associated with synapse formation are determined and wherein the one or more markers are determined in the effector cells and/or in the target cells.

9. The method in accordance with claim 8, wherein the marker associated with synapse formation is selected from the group consisting of calcium signalling markers; spatial clustering of synapse localized molecules such as LFA-1, CD28, CD3, and Agrin; changes to internal cell structure and/or cytoskeleton such as F-Actin, Talin, microtubules, centrosome, lytic granules, nucleus position, and mitochondrial relocation; changes in effector cell motility; changes in external cell morphology and/or cell shape; and apoptosis of target cells.

10. The method in accordance with claim 1, wherein the markers are determined with sequencing or staining of a marker.

11. The method in accordance with claim 1, wherein the applied force is a force ramp, preferably a linear force ramp.

12. The method in accordance with any one of claim 12, wherein the applied force is an acoustic force, a shear flow force or an acceleration force.

13. The method in accordance with claim 1, wherein after the step of applying a force in step d) the cells are resuspended and a differential force is applied such that formed aspecific cell-cell bonds are broken.

14. The method in accordance with claim 1, wherein said attachment of cells is optional, and, in the step of applying the force away, instead a differential force is applied.

15. Method in accordance with claim 14, wherein the differential force is such that cells that are bound to each other via a synapse remain bound to each other and formed aspecific cell-cell bonds are broken.
